(19) United States
(12) Patent Application Publication
(10) Pub. No.: US 2017/0112824 A1
(43) Pub. Date: Apr. 27, 2017

(54) COMBINATION THERAPIES FOR THE TREATMENT OF FUNGAL INFECTIONS

(71) Applicant: Wright State University, Dayton, OH (US)

(72) Inventors: Yong-Jie Xu, Beavercreek, OH (US); Amanpreet Singh, Fairborn, OH (US)

(21) Appl. No.: 15/332,292

(22) Filed: Oct. 24, 2016

Related U.S. Application Data
(60) Provisional application No. 62/245,310, filed on Oct. 23, 2015.

(51) Int. Cl. A61K 31/4745; A61K 31/17; A61K 31/4174; A61K 31/4196; A61K 31/496; A61K 45/06; A61K 31/137

(52) U.S. Cl. CPC A61K 31/4745; A61K 45/06; A61K 31/17; A61K 31/137; A61K 31/4196; A61K 31/496; A61K 31/4174

(57) ABSTRACT

Methods and pharmaceutical compositions for treating fungal infections in a patient via administration of hydroxyurea and an ergosterol synthesis inhibitor, hydroxyurea and a heme synthesis inhibitor, an ergosterol synthesis inhibitor and a heme synthesis inhibitor, or hydroxyurea, an ergosterol synthesis inhibitor, and a heme synthesis inhibitor.

### CROSS-REFERENCE TO RELATED APPLICATIONS

This application claims the benefit of U.S. Provisional Application Ser. No. 62/245,310, filed Oct. 23, 2015.

### FIELD OF THE INVENTION

The present disclosure relates to the field of therapeutic treatment of fungal infections. Specifically, the present disclosure relates to methods and pharmaceutical compositions for treating a fungal infection in a subject in need thereof via administration of hydroxyurea and an ergosterol synthesis inhibitor, hydroxyurea and a heme synthesis inhibitor, an ergosterol synthesis inhibitor and a heme synthesis inhibitor, or hydroxyurea, an ergosterol synthesis inhibitor, and a heme synthesis inhibitor.

### BACKGROUND

Fungal diseases are a global public health problem. About 1.2 billion people worldwide are estimated to suffer from a fungal disease. Most are infections of the skin, nails, or mucosa, but a substantial minority, particularly those who have weakened immune systems such as the patients who have cancer or HIV/AIDS, is invasive or chronic and difficult to treat. It is estimated that 1.5 to 2 million people die of a fungal infection each year. Most of this mortality is caused by species belonging to four genera of fungi: Aspergillus, Candida, Cryptococcus, and Pneumocystis. Although effective anti-fungals such as the second-generation azoles (ergosterol biosynthesis inhibitors), echinocandins (cell wall synthesis inhibitor), and amphotericin B (membrane disruption) are available, the toxicity of these drugs and the increasing alarm of drug resistance hamper the effective treatment of fungal infections. Accordingly, there remains a need in the art for new pharmaceutical compositions and methods of using the same that can effectively treat fungal infections.

### SUMMARY

Accordingly, the presently disclosed subject matter relates to new pharmaceutical compositions and methods of using the same that can effectively treat fungal infections.

One embodiment of the presently-disclosed subject matter is directed to a method of treating a fungal infection in a subject in need thereof, the method comprising administering to the subject a therapeutically effective amount of hydroxyurea and a therapeutically effective amount of an ergosterol synthesis inhibitor.

Another embodiment of the presently-disclosed subject matter is directed to a pharmaceutical composition comprising a therapeutically effective amount of hydroxyurea, a therapeutically effective amount of an ergosterol synthesis inhibitor, and a pharmaceutically-acceptable excipient.

A further embodiment of the presently-disclosed subject matter is directed to a method of treating a fungal infection in a subject in need thereof, the method comprising administering to the subject a therapeutically effective amount of hydroxyurea and a therapeutically effective amount of a heme synthesis inhibitor.

An additional embodiment of the presently-disclosed subject matter is directed to a pharmaceutical composition comprising a therapeutically effective amount of hydroxyurea, a therapeutically effective amount of a heme synthesis inhibitor, and a pharmaceutically-acceptable excipient.

One embodiment of the presently-disclosed subject matter is directed to a method of treating a fungal infection in a subject in need thereof, the method comprising administering to the subject a therapeutically effective amount of an ergosterol synthesis inhibitor and a therapeutically effective amount of a heme synthesis inhibitor.

Another embodiment of the presently-disclosed subject matter is directed to a pharmaceutical composition comprising a therapeutically effective amount of an ergosterol synthesis inhibitor, a therapeutically effective amount of a heme synthesis inhibitor, and a pharmaceutically-acceptable excipient.

An additional embodiment of the presently-disclosed subject matter is directed to a method of treating a fungal infection in a subject in need thereof, the method comprising administering to the subject a therapeutically effective amount of hydroxyurea, a therapeutically effective amount of an ergosterol synthesis inhibitor, and a therapeutically effective amount of a heme synthesis inhibitor.

A further embodiment of the presently-disclosed subject matter is directed to a pharmaceutical composition comprising a therapeutically effective amount of hydroxyurea, a therapeutically effective amount of an ergosterol synthesis inhibitor, a therapeutically effective amount of a heme synthesis inhibitor, and a pharmaceutically-acceptable excipient.

### BRIEF DESCRIPTION OF THE DRAWINGS

FIG. 1. Standard spot assay to test the cell-killing effect of hydroxyurea (HU) and other agents at the indicated concentrations. Serial dilutions of logarithmically growing wild type Schizosaccharomyces pombe were spotted on plates containing no drug (top left), hydroxyurea, the heme synthesis inhibitor sampangine, or the ergosterol synthesis inhibitors itraconazole, terbinafine, and clotrimazole, and combinations of hydroxyurea with the indicated agents. The plates were incubated at 30° C. for three days and then photographed. Each individual drug has no or minimal effect on cell growth. However, combining hydroxyurea with sampangine or the ergosterol inhibitors together showed a synergistic effect on suppressing the cell growth.

FIG. 2A-C. Combination of hydroxyurea (HU) and sampangine (SMP) shows synergistic cell-killing effect on wildtype Schizosaccharomyces pombe. FIG. 2A is a graphical representation of the cell-killing effect of hydroxyurea, sampangine, and various combinations of the two drugs. FIG. 2B shows the combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 2C is a combination index plot, wherein Fa values on the X axis are the values of cell growth inhibition at various CI values. Values above the horizontal line, on the line, and below the line represent antagonistic, additive, and synergistic effect, respectively.

FIG. 3A-C. The cell-killing effect of hydroxyurea (HU) and sampangine (SMP) on wildtype Saccharomyces cerevisiae. FIG. 3A is a graphical representation of the cell-killing effect of hydroxyurea, sampangine, and various combinations of the two drugs. FIG. 3B shows the combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 3C is a combination index plot, wherein Fa values on the X axis are the values of cell growth inhibition at various CI values. Values above the horizontal line, on the line, and below the line represent antagonistic, additive, and synergistic effect, respectively.

FIG. 4A-C. The cell-killing effect of hydroxyurea (HU) and sampangine (SMP) on pathogenic fungus Candida albicans. FIG. 4A is a graphical representation of the cell-killing effect of hydroxyurea, sampangine, and various combinations of the two drugs. FIG. 4B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 4C is a combination index plot, wherein Fa values on the X axis are the values of cell growth inhibition at various CI values. Values above the horizontal line, on the line, and below the line represent antagonistic, additive, and synergistic effect, respectively.

FIG. 5A-C. The cell-killing effect of hydroxyurea (HU) and ergosterol synthesis inhibitor ketoconazole (Keto) on pathogenic fungus Candida albicans. FIG. 5A is a graphical representation of the cell-killing effect of hydroxyurea, ketoconazole, and various combinations of the two drugs. FIG. 5B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 5C is a combination index plot, wherein Fa values on the X axis are the values of cell growth inhibition at various CI values. Values above the horizontal line, on the line, and below the line represent antagonistic, additive, and synergistic effect, respectively.

FIG. 6A-C. The cell-killing effect of hydroxyurea (HU) and ergosterol synthesis inhibitor clotrimazole (Clot) on pathogenic fungus Candida albicans. FIG. 6A is a graphical representation of the cell-killing effect of hydroxyurea, clotrimazole, and various combination of the two drugs. FIG. 6B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 6C is a combination index plot. Fa values on the X axis are the values of cell growth inhibition at various CI values. Values above the horizontal line, on the line, and below the line represent antagonistic, additive, and synergistic effect, respectively.

FIG. 7A-C. The cell-killing effect of hydroxyurea (HU) and ergosterol synthesis inhibitor terbinafine (Terb) on pathogenic fungus Candida albicans. FIG. 7A is a graphical representation of the cell-killing effect of hydroxyurea, terbinafine, and various combinations of the two drugs. FIG. 7B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 7C is a combination index plot, wherein Fa values on the X axis are the values of cell growth inhibition at various CI values. Values above the horizontal line, on the line, and below the line represent antagonistic, additive, and synergistic effect, respectively.

FIG. 8A-C. The cell-killing effect of hydroxyurea (HU) and ergosterol synthesis inhibitor itraconazole (Itra) on pathogenic fungus Candida albicans. FIG. 8A is a graphical representation of the cell-killing effect of hydroxyurea, intraconazole, and various combinations of the two drugs. FIG. 8B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 8C is a combination index plot, wherein Fa values on the X axis are the values of cell growth inhibition at various CI values. Values above the horizontal line, on the line, and below the line represent antagonistic, additive, and synergistic effect, respectively.

FIG. 9A-C. Combination of hydroxyurea (HU) and ergosterol synthesis inhibitor clotrimazole (Clot) shows synergistic cell-killing effect on wildtype Saccharomyces cerevisiae. FIG. 9A is a graphical representation of the cell-killing effect of hydroxyurea, clotrimazole, and various combinations of the two drugs. FIG. 9B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 9C is a combination index plot, wherein Fa values on the X axis are the values of cell growth inhibition at various CI values. Values above the horizontal line, on the line, and below the line represent antagonistic, additive, and synergistic effect, respectively.

FIG. 10A-C. The cell-killing effect of hydroxyurea (HU) and ergosterol synthesis inhibitor fluconazole (Fuco) on wildtype Saccharomyces cerevisiae. FIG. 10A is a graphical representation of the cell-killing effect of hydroxyurea, fluconazole, and various combinations of the two drugs. FIG. 10B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 10C is a combination index plot, wherein Fa values on the X axis are the values of cell growth inhibition at various CI values. Values above the horizontal line, on the line, and below the line represent antagonistic, additive, and synergistic effect, respectively.

FIG. 11A-C. The cell-killing effect of hydroxyurea (HU) and ergosterol synthesis inhibitor itraconazole (Itra) on wildtype Saccharomyces cerevisiae. FIG. 11A is a graphical representation of the cell-killing effect of hydroxyurea, intraconazole, and various combinations of the two drugs. FIG. 11B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 11C is a combination index plot, wherein Fa values on the X axis are the values of cell growth inhibition at various CI values. Values above the horizontal line, on the line, and below the line represent antagonistic, additive, and synergistic effect, respectively.

FIG. 12A-C. The cell-killing effect of hydroxyurea (HU) and ergosterol synthesis inhibitor ketoconazole (keto) on wildtype Saccharomyces cerevisiae. FIG. 12A is a graphical representation of the cell-killing effect of HU, ketoconazole, and various combinations of the two drugs. FIG. 12B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 12C is a combination index plot, wherein Fa values on the X axis are the values of cell growth inhibition at various CI values. Values above the horizontal line, on the line, and below the line represent antagonistic, additive, and synergistic effect, respectively.

FIG. 13A-C. The cell-killing effect of hydroxyurea (HU) and ergosterol synthesis inhibitor terbinafine (Terb) on wildtype Saccharomyces cerevisiae. FIG. 13A is a graphical representation of the cell-killing effect of hydroxyurea, terbinafine, and various combinations of the two drugs. FIG. 13B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 13C is a combination index plot, wherein Fa values on the X axis are the values of cell growth inhibition at various CI values. Values above the horizontal line, on the line, and below the line represent antagonistic, additive, and synergistic effect, respectively.

FIG. 14A-C. Combination of hydroxyurea (HU) and ergosterol synthesis inhibitor clotrimazole (Clot) shows synergistic cell-killing effect on wildtype Schizosaccharomyces pombe. FIG. 14A is a graphical representation of the cell-killing effect of hydroxyurea, clotrimazole, and various combinations of the two drugs. FIG. 14B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 14C is a combination index plot, wherein Fa values on the X axis are the values of cell growth inhibition at various CI values. Values above the horizontal line, on the line, and below the line represent antagonistic, additive, and synergistic effect, respectively.

FIG. 15A-C. The cell-killing effect of hydroxyurea (HU) and ergosterol synthesis inhibitor fluconazole (Fuco) on wildtype Schizosaccharomyces pombe. FIG. 15A is a graphical representation of the cell-killing effect of hydroxyurea, fluconazole, and various combinations of the two drugs. FIG. 15B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 15C is a combination index plot, wherein Fa values on the X axis are the values of cell growth inhibition at various CI values. Values above the horizontal line, on the line, and below the line represent antagonistic, additive, and synergistic effect, respectively.

FIG. 16A-C. The cell-killing effect of hydroxyurea (HU) and ergosterol synthesis inhibitor itraconazole (itra) on wildtype Schizosaccharomyces pombe. FIG. 16A is a graphical representation of the cell-killing effect of hydroxyurea, intraconazole, and various combinations of the two drugs. FIG. 16B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 16C is a combination index plot, wherein Fa values on the X axis are the values of cell growth inhibition at various CI values. Values above the horizontal line, on the line, and below the line represent antagonistic, additive, and synergistic effect, respectively.

FIG. 17A-C. The cell-killing effect of hydroxyurea (HU) and ergosterol synthesis inhibitor ketoconazole (Keto) on wildtype Schizosaccharomyces pombe. FIG. 17A is a graphical representation of the cell-killing effect of hydroxyurea, ketoconazole, and various combinations of the two drugs. FIG. 17B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 17C is a combination index plot, wherein Fa values on the X axis are the values of cell growth inhibition at various CI values. Values above the horizontal line, on the line, and below the line represent antagonistic, additive, and synergistic effect, respectively.

FIG. 18A-C. The cell-killing effect of hydroxyurea (HU) and ergosterol synthesis inhibitor terbinafine (Terb) on wildtype Schizosaccharomyces pombe. FIG. 18A is a graphical representation of the cell-killing effect of hydroxyurea, terbinafine, and various combinations of the two drugs. FIG. 18B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 18C is a combination index plot, wherein Fa values on the X axis are the values of cell growth inhibition at various CI values. Values above the horizontal line, on the line, and below the line represent antagonistic, additive, and synergistic effect, respectively.

FIG. 19A-C. Combination of sampangine (SMP) and ergosterol synthesis inhibitor clotrimazole (Clot) shows synergistic cell-killing effect on pathogenic fungus Candida albicans. FIG. 19A is a graphical representation of the cell-killing effect of sampangine, clotrimazole, and various combinations of the two drugs. FIG. 19B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 19C is a combination index plot, wherein Fa values on the X axis are the values of cell growth inhibition at various CI values. Values above the horizontal line, on the line, and below the line represent antagonistic, additive, and synergistic effect, respectively.

FIG. 20A-C. The cell-killing effect of sampangine (SMP) and ergosterol synthesis inhibitor itraconazole (Itra) on pathogenic fungus Candida albicans. FIG. 20A is a graphical representation of the cell-killing effect of sampangine, intraconazole, and various combinations of the two drugs. FIG. 20B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 20C is a combination index plot, wherein Fa values on the X axis are the values of cell growth inhibition at various CI values. Values above the horizontal line, on the line, and below the line represent antagonistic, additive, and synergistic effect, respectively.

FIG. 21A-C. The cell-killing effect of sampangine (SMP) and ergosterol synthesis inhibitor ketoconazole (Keto) on pathogenic fungus Candida albicans. FIG. 21A is a graphical representation of the cell-killing effect of sampangine, ketoconazole, and various combinations of the two drugs. FIG. 21B shows combination index (CI) values of the drug combinations. CI values of less than 0.1 indicate a very strong synergism, CI values between 0.1 to 0.3 indicate a strong synergism, CI values between 0.3 and 0.7 indicate moderate synergism, and CI values between 0.7 to 0.85 indicate a slight synergism. FIG. 21C is a combination index plot, wherein Fa values on the X axis are the values of cell growth inhibition at various CI values. Values above the horizontal line, on the line, and below the line represent antagonistic, additive, and synergistic effect, respectively.

### DETAILED DESCRIPTION

Particular details of various embodiments of the invention are set forth to illustrate certain aspects and not to limit the scope of the invention. It will be apparent to one of ordinary skill in the art that modifications and variations are possible without departing from the scope of the embodiments defined in the appended claims. More specifically, although some aspects of embodiments of the present invention may be identified herein as preferred or particularly advantageous, it is contemplated that the embodiments of the present invention are not limited to these preferred aspects.

Unless defined otherwise, all technical and scientific terms used herein have the same meaning as commonly understood by one of ordinary skill in the art to which the presently-disclosed subject matter belongs.

The presently disclosed data demonstrates a new concept of anti-fungal pharmaceutical compositions and methods that can effectively treat fungal infections. This new concept of anti-fungal pharmaceutical compositions and methods for treating fungal infections in a patient includes administration of hydroxyurea and an ergosterol synthesis inhibitor, hydroxyurea and a heme synthesis inhibitor, an ergosterol synthesis inhibitor and a heme synthesis inhibitor, or hydroxyurea, an ergosterol synthesis inhibitor, and a heme synthesis inhibitor.

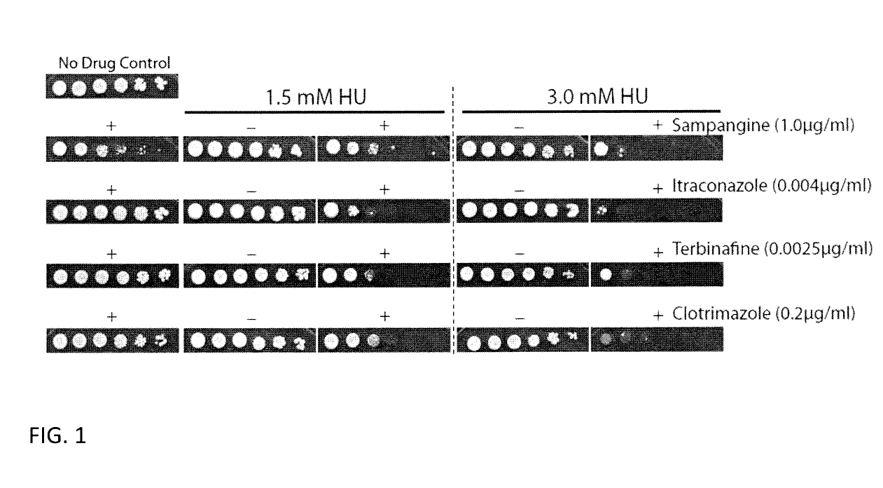

The present investigators recently screened several mutants in the fission yeast Schizosaccharomyces pombe, a non-pathogenic fungus, that are highly sensitive to hydroxyurea, an inhibitor of ribonucleotide reductase that blocks DNA replication. Since the mutations cause defects in either ergosterol or heme synthesis, it was hypothesized that inhibition of the synthesis pathways may mimic the effects of the mutations in wild type cells and thus sensitize the cells to hydroxyurea. To test this idea, the present investigators performed a spot assay on plates containing the inhibitors of heme or ergosterol synthesis, hydroxyurea, or both, and monitored the drug effects on cell growth of Schizosaccharomyces pombe (FIG. 1). The investigators determined that all three tested ergosterol synthesis inhibitors (clotrimazole, terbinafine, and itraconazole) and the heme synthesis inhibitor sampangine demonstrate a synergistic effect when used in combination with hydroxyurea in suppressing cell growth.

To analyze the synergistic drug effect in further detail, the present investigators measured the cytotoxicity in 96 well plates. The present investigators tested the combination of hydroxyurea and ergosterol synthesis inhibitors, hydroxyurea and heme synthesis inhibitors, and ergosterol synthesis inhibitors and heme synthesis inhibitors. The dose response curve of each drug was obtained in order to determine the drug concentration required to suppress 50% cell growth (IC50 value). The drug concentrations showing minimal effects on cell growth were then chosen for various drug combinations. The synergistic, additive, and antagonistic effects between the combined drugs were determined by using the Chou and Talalay method (Chou, T C (2006) Theoretical basis, experimental design, and computerized stimulation of synergism and antagonism in drug combination studies. Pharmacol. Rev. 58:621-681). Based on the presently-disclosed data, the combinations of hydroxyurea and ergosterol synthesis inhibitors, hydroxyurea and heme synthesis inhibitors, and ergosterol synthesis inhibitors and heme synthesis inhibitors can be used for novel anti-fungal pharmaceutical compositions and methods that can effectively treat fungal infections.

Accordingly, the presently-disclosed subject matter relates to methods and pharmaceutical compositions for treating a fungal infection in a patient via administration of hydroxyurea and an ergosterol synthesis inhibitor, hydroxyurea and a heme synthesis inhibitor, an ergosterol synthesis inhibitor and a heme synthesis inhibitor, or hydroxyurea, an ergosterol synthesis inhibitor, and a heme synthesis inhibitor.

In some embodiments, the presently-disclosed subject matter includes a method of treating a fungal infection in a subject in need thereof via administration of: a therapeutically effective amount of hydroxyurea and a therapeutically effective amount of an ergosterol synthesis inhibitor; a therapeutically effective amount of hydroxyurea and a therapeutically effective amount of a heme synthesis inhibitor; a therapeutically effective amount of an ergosterol synthesis inhibitor and a therapeutically effective amount of a heme synthesis inhibitor; or a therapeutically effective amount of hydroxyurea, a therapeutically effective amount of an ergosterol synthesis inhibitor, and a therapeutically effective amount of a heme synthesis inhibitor.

As used herein, the term “treating” relates to any treatment of a fungal infection, including but not limited to prophylactic treatment and therapeutic treatment. “Treating” includes any effect, e.g., lessening, reducing, modulating, or eliminating, that results in the improvement of the fungal infection. “Treating” or “treatment” of a fungal infection includes: inhibiting the fungal infection, i.e., arresting the development of the fungal infection or its clinical symptoms; or relieving the fungal infection, i.e., causing temporary or permanent regression of the fungal infection or its clinical symptoms.

A “subject” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like).

An “effective amount” as defined herein in relation to the treatment of a fungal infection is an amount that will decrease, reduce, inhibit, or otherwise abrogate the growth of a fungal cell. An effective amount as used herein also includes an amount sufficient to delay the development of a symptom of a fungal infection, alter the course of a fungal infection (for example but not limited to, slow the progression of a symptom of the fungal infection, such as growth of the fungal population), or reverse a symptom of the fungal infection. The “effective amount” will vary depending on the fungal infection and its severity and the age, weight, etc., of the mammal to be treated. Additionally, the dosage can vary depending upon the dosage form employed and the route of administration utilized.

In embodiments of a method of treating a fungal infection in subject in need thereof, the compounds of the combination therapy (including a therapeutically effective amount of hydroxyurea and a therapeutically effective amount of an ergosterol synthesis inhibitor, a therapeutically effective amount of hydroxyurea and a therapeutically effective amount of a heme synthesis inhibitor, a therapeutically effective amount of an ergosterol synthesis inhibitor and a therapeutically effective amount of a heme synthesis inhibitor, or a therapeutically effective amount of hydroxyurea, a therapeutically effective amount of an ergosterol synthesis inhibitor, and a therapeutically effective amount of a heme synthesis inhibitor) can be administered simultaneously. In other embodiments of a method of treating a fungal infection in a subject in need thereof, the compounds of the combination therapy can be administered sequentially. In some embodiments of a method of a treating a fungal infection in a subject in need thereof, the compounds of the combination therapy can be administered independently by the same route or by two or more different routes of administration, depending on the dosage forms employed.

Exemplary modes of administration of the compounds of the combination therapy include, but are not limited to, injection, infusion, inhalation (e.g., intranasal or intratracheal), ingestion, rectal, and topical (including buccal and sublingual) administration. Local administration can be used if, for example, extensive side effects or toxicity is associated with the compounds of the combination therapy, and to, for example, permit a high localized concentration of the compounds to the infection site. Administration to deliver compounds of the combination therapy systemically or to a desired surface or target can include, but is not limited to, injection, infusion, instillation, and inhalation administration. Injection includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The compounds in the particular combination therapy being used to treat a fungal infection in a subject can determine the mode of administration to be used.

In some embodiments, a method of treating a fungal infection in a subject in need thereof comprises administering to the subject a therapeutically effective amount of hydroxyurea and a therapeutically effective amount of an ergosterol synthesis inhibitor.

In certain embodiments of a method of treating a fungal infection in a subject in need thereof comprising administering to the subject a therapeutically effective amount of hydroxyurea and a therapeutically effective amount of an ergosterol synthesis inhibitor, the fungal infection involves a fungal species comprising Candida, Aspergillus, Cryptococcus, Pneumocystis, Histoplasma, and Cryptococcus species. In some embodiments, the fungal species is Candida albicans. In certain embodiments, the fungal species is Aspergillus fumigatus. In other embodiments, the fungal species is Histoplasma capsulatum. In some embodiments, the fungal species is Cryptococcus neoformans.

In some embodiments of a method of treating a fungal infection in a subject in need thereof comprising administering to the subject a therapeutically effective amount of hydroxyurea and a therapeutically effective amount of an ergosterol synthesis inhibitor, the ergosterol synthesis inhibitor comprises an azole. In other embodiments, the ergosterol synthesis inhibitor comprises ketoconazole. In further embodiments, the ergosterol synthesis inhibitor comprises clotrimazole. In certain embodiments, the ergosterol synthesis inhibitor comprises itraconazole. In even further embodiments, the ergosterol synthesis inhibitor comprises terbinafine. In some embodiments, the ergosterol synthesis inhibitor comprises fluconazole.

In certain embodiments, a method of treating a fungal infection in a subject in need thereof comprises administering to the subject a therapeutically effective amount of hydroxyurea and a therapeutically effective amount of a heme synthesis inhibitor. In certain embodiments, the heme synthesis inhibitor comprises sampangine. In some embodiments, the fungal infection involves a fungal species comprising Candida, Aspergillus, Cryptococcus, Pneumocystis, Histoplasma, and Cryptococcus species. In some embodiments, the fungal species is Candida albicans. In certain embodiments, the fungal species is Aspergillus fumigatus. In other embodiments, the fungal species is Histoplasma capsulatum. In some embodiments, the fungal species is Cryptococcus neoformans.

In some embodiments, a method of treating a fungal infection in a subject in need thereof comprises administering to the subject a therapeutically effective amount of an ergosterol synthesis inhibitor and a therapeutically effective amount of a heme synthesis inhibitor. In some embodiments, the fungal infection involves a fungal species comprising Candida, Aspergillus, Cryptococcus, Pneumocystis, Histoplasma, and Cryptococcus species. In some embodiments, the fungal species is Candida albicans. In certain embodiments, the fungal species is Aspergillus fumigatus. In other embodiments, the fungal species is Histoplasma capsulatum. In some embodiments, the fungal species is Cryptococcus neoformans.

In some embodiments of a method of treating a fungal infections in a subject in need thereof comprising administering to the subject a therapeutically effective amount of an ergosterol synthesis inhibitor and a therapeutically effective amount of a heme synthesis inhibitor, the heme synthesis inhibitor comprises sampangine. In some embodiments, the ergosterol synthesis inhibitor comprises an azole. In other embodiments, the ergosterol synthesis inhibitor comprises ketoconazole. In further embodiments, the ergosterol synthesis inhibitor comprises clotrimazole. In certain embodiments, the ergosterol synthesis inhibitor comprises itraconazole. In even further embodiments, the ergosterol synthesis inhibitor comprises terbinafine. In some embodiments, the ergosterol synthesis inhibitor comprises fluconazole.

In other embodiments, a method of treating a fungal infections in a subject in need thereof comprises administering to the subject a therapeutically effective amount of hydroxyurea, a therapeutically effective amount of an ergosterol synthesis inhibitor, and a therapeutically effective amount of a heme synthesis inhibitor. In some embodiments, the fungal infection involves a fungal species comprising Candida, Aspergillus, Cryptococcus, Pneumocystis, Histoplasma, and Cryptococcus species. In some embodiments, the fungal species is Candida albicans. In certain embodiments, the fungal species is Aspergillus fumigatus. In other embodiments, the fungal species is Histoplasma capsulatum. In some embodiments, the fungal species is Cryptococcus neoformans.

In some embodiments of a method of treating a fungal infection in a subject in need thereof comprising administering to the subject a therapeutically effective amount of hydroxyurea, a therapeutically effective amount of an ergosterol synthesis inhibitor, and a therapeutically effective amount of a heme synthesis inhibitor, the heme synthesis inhibitor comprises sampangine. In some embodiments, the ergosterol synthesis inhibitor comprises an azole. In other embodiments, the ergosterol synthesis inhibitor comprises ketoconazole. In further embodiments, the ergosterol synthesis inhibitor comprises clotrimazole. In certain embodiments, the ergosterol synthesis inhibitor comprises itraconazole. In even further embodiments, the ergosterol synthesis inhibitor comprises terbinafine. In some embodiments, the ergosterol synthesis inhibitor comprises fluconazole.

In some embodiments, the presently-disclosed subject matter includes a pharmaceutical composition comprising: a therapeutically effective amount of hydroxyurea, a therapeutically effective amount of an ergosterol synthesis inhibitor, and a pharmaceutically-acceptable excipient; a therapeutically effective amount of hydroxyurea, a therapeutically effective amount of a heme synthesis inhibitor, and a pharmaceutically-acceptable excipient; a therapeutically effective amount of an ergosterol synthesis inhibitor, a therapeutically effective amount of a heme synthesis inhibitor, and a pharmaceutically-acceptable excipient; or a therapeutically effective amount of hydroxyurea, a therapeutically effective amount of an ergosterol synthesis inhibitor, a therapeutically effective amount of a heme synthesis inhibitor, and a pharmaceutically-acceptable excipient.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Thus, the term “pharmaceutical excipient” is used herein to describe any ingredient other than hydroxyurea, the ergosterol synthesis inhibitors, and the heme synthesis inhibitors compound(s) of the present disclosure. Examples of pharmaceutical excipients include one or more substances which may act as carriers, diluents, flavoring agents, solubilizers, lubricants, suspending agents, binders, preservatives, wetting agents, tablet disintegrating agents, or an encapsulating material. The choice of excipient will to a large extent depend on factors such as the particular mode of administration, the effect of the excipient on solubility and/or stability, and the nature of the dosage form as is understood by those of skill in the art. A “pharmaceutical excipient” includes both one and more than one such excipient.

In some embodiments, the compounds of the pharmaceutical composition (including: a therapeutically effective amount of hydroxyurea, a therapeutically effective amount of an ergosterol synthesis inhibitor, and a pharmaceutically-acceptable excipient; a therapeutically effective amount of hydroxyurea, a therapeutically effective amount of a heme synthesis inhibitor, and a pharmaceutically-acceptable excipient; a therapeutically effective amount of an ergosterol synthesis inhibitor, a therapeutically effective amount of a heme synthesis inhibitor, and a pharmaceutically-acceptable excipient; or a therapeutically effective amount of hydroxyurea, a therapeutically effective amount of an ergosterol synthesis inhibitor, a therapeutically effective amount of a heme synthesis inhibitor, and a pharmaceutically-acceptable excipient), can be formulated for administration to a subject in solid, liquid, or gel form. This can include formulation for: (1) parenteral administration, for example, by subcutaneous, intramuscular, intravenous or epidural injection as, for example, a sterile solution or suspension, or sustained-release formulation; (2) topical application, for example, as a cream, ointment, creams, lotions, ointments, gels, shampoos, sprays, aerosols, solutions, emulsions, a controlled-release patch applied to the skin, and other forms known to one of skill in the art; (3) oral administration, for example, drenches (aqueous or non-aqueous solutions or suspensions), lozenges, capsules, pills, tablets, boluses, powders, granules, and pastes; (4) intravaginally or intrarectally, for example, as a pessary, cream or foam; (5) sublingually; (6) ocularly; (7) transdermally; (8) transmucosally; or (9) nasally. As such, examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquids such as suspensions (e.g., aqueous or non-aqueous liquid suspensions, oil-in-water emulsions, or water-in-oil liquid emulsions), solutions, and elixirs; and sterile solids (e.g., crystalline or amorphous solids) that can be reconstituted to provide liquid dosage forms.

In some embodiments, the pharmaceutical composition comprises a therapeutically effective amount of hydroxyurea, a therapeutically effective amount of an ergosterol synthesis inhibitor, and a pharmaceutically-acceptable excipient.

In some embodiments of the pharmaceutical composition comprising a therapeutically effective amount of hydroxyurea, a therapeutically effective amount of an ergosterol synthesis inhibitor, and a pharmaceutically-acceptable excipient, the ergosterol synthesis inhibitor comprises an azole. In other embodiments, the ergosterol synthesis inhibitor comprises ketoconazole. In further embodiments, the ergosterol synthesis inhibitor comprises clotrimazole. In certain embodiments, the ergosterol synthesis inhibitor comprises itraconazole. In even further embodiments, the ergosterol synthesis inhibitor comprises terbinafine. In some embodiments, the ergosterol synthesis inhibitor comprises fluconazole.

Another embodiment of the presently-disclosed subject matter is directed to a pharmaceutical composition comprising a therapeutically effective amount of hydroxyurea, a therapeutically effective amount of a heme synthesis inhibitor, and a pharmaceutically-acceptable excipient. In certain embodiments, the heme synthesis inhibitor comprises sampangine.

A further embodiment of the presently-disclosed subject matter is directed to a pharmaceutical composition comprising a therapeutically effective amount of an ergosterol synthesis inhibitor, a therapeutically effective amount of a heme synthesis inhibitor, and a pharmaceutically-acceptable excipient. In some embodiments, the ergosterol synthesis inhibitor comprises an azole. In other embodiments, the ergosterol synthesis inhibitor comprises ketoconazole. In further embodiments, the ergosterol synthesis inhibitor comprises clotrimazole. In certain embodiments, the ergosterol synthesis inhibitor comprises itraconazole. In even further embodiments, the ergosterol synthesis inhibitor comprises terbinafine. In some embodiments, the ergosterol synthesis inhibitor comprises fluconazole. In certain embodiments, the heme synthesis inhibitor comprises sampangine.

An additional embodiment of the presently-disclosed subject matter is directed to a pharmaceutical composition comprising a therapeutically effective amount of hydroxyurea, a therapeutically effective amount of an ergosterol synthesis inhibitor, a therapeutically effective amount of a heme synthesis inhibitor, and a pharmaceutically-acceptable excipient. In some embodiments, the ergosterol synthesis inhibitor comprises an azole. In other embodiments, the ergosterol synthesis inhibitor comprises ketoconazole. In further embodiments, the ergosterol synthesis inhibitor comprises clotrimazole. In certain embodiments, the ergosterol synthesis inhibitor comprises itraconazole. In even further embodiments, the ergosterol synthesis inhibitor comprises terbinafine. In some embodiments, the ergosterol synthesis inhibitor comprises fluconazole. In certain embodiments, the heme synthesis inhibitor comprises sampangine.

### EXAMPLES

The following examples are given by way of illustration and are in no way intended to limit the scope of the presently-disclosed subject matter.

### Example 1

Materials and Methods

Spot assay: To test the drug sensitivity on plates, 2×10⁷ cells/ml of logarithmically growing Schizosaccharomyces pombe were diluted in 5-fold steps and spotted onto YE6S plates containing hydroxyurea (HU), sampangine (SMP), ergosterol synthesis inhibitors, both hydroxyurea and sampangine, or both hydroxyurea and an ergosterol synthesis inhibitor at the indicated concentrations. The plates were incubated at 30° C. for 3 days and then photographed.

Results and Discussion

The present investigators performed a spot assay on plates containing a heme synthesis inhibitor, an ergosterol synthesis inhibitor, hydroxyurea, both a heme synthesis inhibitor and hydroxyurea, or both an ergosterol synthesis inhibitor and hydroxyurea, and monitored the drug effects on cell growth of Schizosaccharomyces pombe (FIG. 1). The inventors unexpectedly found that all three tested ergosterol synthesis inhibitors (clotrimazole, terbinafine, and itraconazole) and the heme synthesis inhibitor sampangine (SMP) have a synergistic effect in suppressing cell growth when used in combination with hydroxyurea.

### Example 2

Materials and Methods

96 well plates assay: To assess the cell-killing effects of inhibitors of heme synthesis, inhibitors of ergosterol synthesis, hydroxyurea, and their various combinations, logarithmically growing Schizosaccharomycs pombe, Saccharomyces cerevisiae, and the pathogenic fungus Candida albicans cells were inoculated on 94 well plates at 3000 cells/well. Various drugs and their combinations were then added to the final volume of 200 μl. The same amounts of carriers were added as the control. Cells were incubated at 30° C. for 48 hours. The plates were scanned in a plate reader at A600. The synergistic, additive, or antagonistic effect of the drug combinations, as indicated by the combination index (CI) values, (see Table 1 below), were calculated by using the Chou and Talalay method.

TABLE 1
Combination index values and indication of synergistic,
additive, or antagonistic effect of drug combinations.

| Range of Combination Index | Interpretation |
|---|---|
| <0.1 | Synergism (very strong) |
| 0.1-0.3 | Synergism (strong) |
| 0.3-0.7 | Synergism (moderate) |
| 0.7-0.85 | Synergism (slight) |
| 0.85-1.10 | Additive (nearly) |
| 1.10-1.20 | Antagonism (slight) |
| 1.20-1.45 | Antagonism (moderate) |
| 1.45-3.3 | Antagonism |
| 3.3-10 | Antagonism (strong) |
| >10 | Antagonism (very strong) |

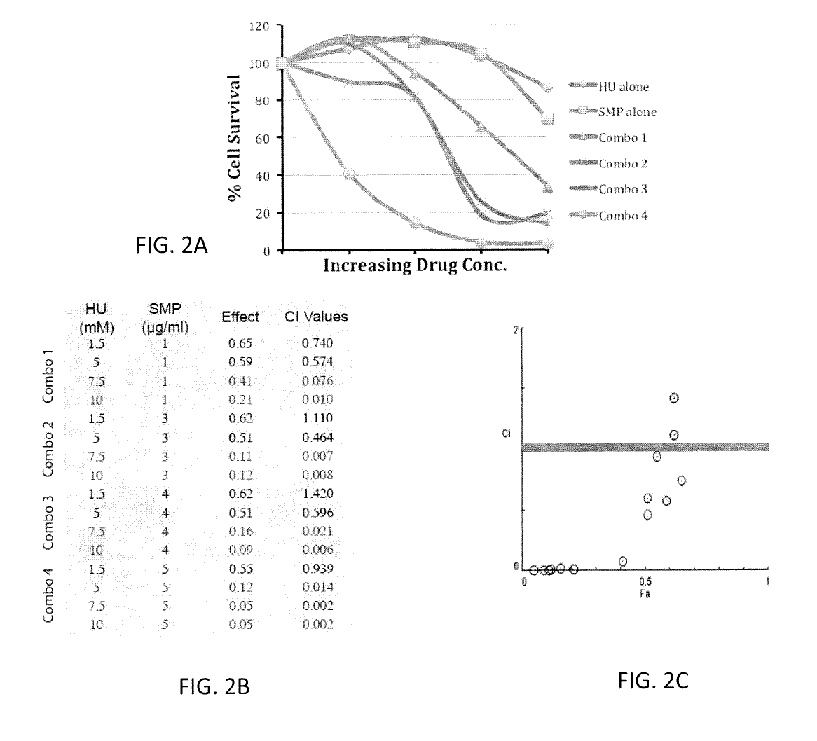

Drug Combinations Used for Testing on Schizosaccharomyces pombe in FIG. 2A-C:
Concentration of hydroxyurea used (mM): 0, 1.5, 5.0, 7.5, 10.0.
Concentration of sampangine used (μg/ml): 0, 1.0, 3.0, 4.0, 5.0.

### Combo 1: (0+0), (1.5+1.0), (5.0+1.0), (7.5+1.0), (10.0+1.0).

### Combo 2: (0+0), (1.5+3.0), (5.0+3.0), (7.5+3.0), (10.0+3.0).

### Combo 3: (0+0), (1.5+4.0), (5.0+4.0), (7.5+4.0), (10.0+4.0).

### Combo 4: (0+0), (1.5+5.0), (5.0+5.0), (7.5+5.0), (10.0+5.0).

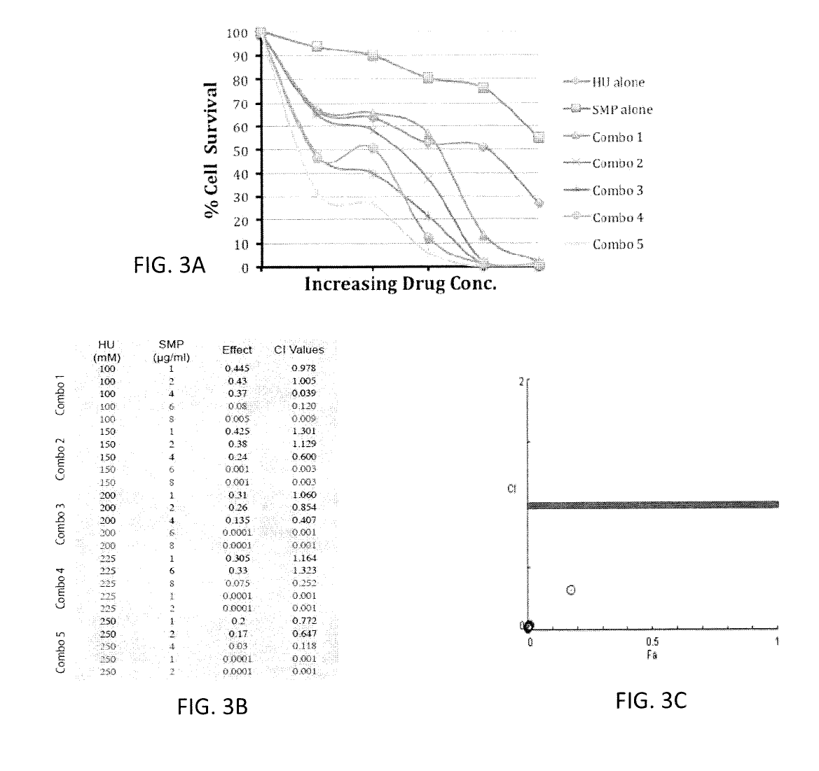

Drug Combinations Used for Testing on Saccharomyces cerevisiae in FIG. 3A-C:
Concentration of hydroxyurea used (mM): 0, 100.0, 150.0, 200.0, 225.0, 250.0.
Concentration of sampangine used (μg/ml): 0, 1.0, 2.0, 4.0, 6.0, 8.0.

### Comb 1: (0+0), (100.0+1.0), (100.0+2.0), (100.0+4.0), (100.0+6.0), (100.0+8.0)

### Comb 2: (0+0), (150.0+1.0), (150.0+2.0), (150.0+4.0), (150.0+6.0), (150.0+8.0)

### Comb 3: (0+0), (200.0+1.0), (200.0+2.0), (200.0+4.0), (200.0+6.0), (200.0+8.0)

### Comb 4: (0+0), (225.0+1.0), (225.0+2.0), (225.0+4.0), (225.0+6.0), (225.0+8.0)

### Comb 5: (0+0), (250.0+1.0), (250.0+2.0), (250.0+4.0), (250.0+6.0), (250.0+8.0)

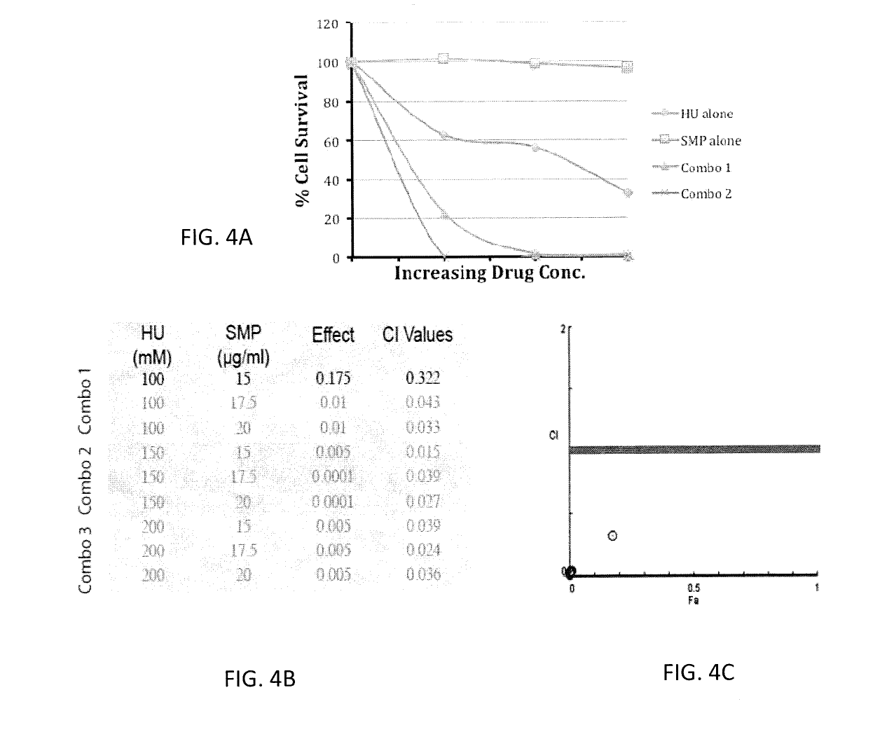

Drug Combinations Used for Testing on Candia albicans in FIG. 4A-C:
Concentration of hydroxyurea used (mM): 0, 100.0, 150.0, 200.0.
Concentration of sampangine used (μg/ml): 0, 15.0, 17.5, 20.0.

### Comb 1: (0+0), (100.0+15.0), (100.0+17.5), (100.0+20.0).

### Comb 2: (0+0), (150.0+15.0), (150.0+17.5), (150.0+20.0).

### Comb 3: (0+0), (200.0+15.0), (200.0+17.5), (200.0+20.0).

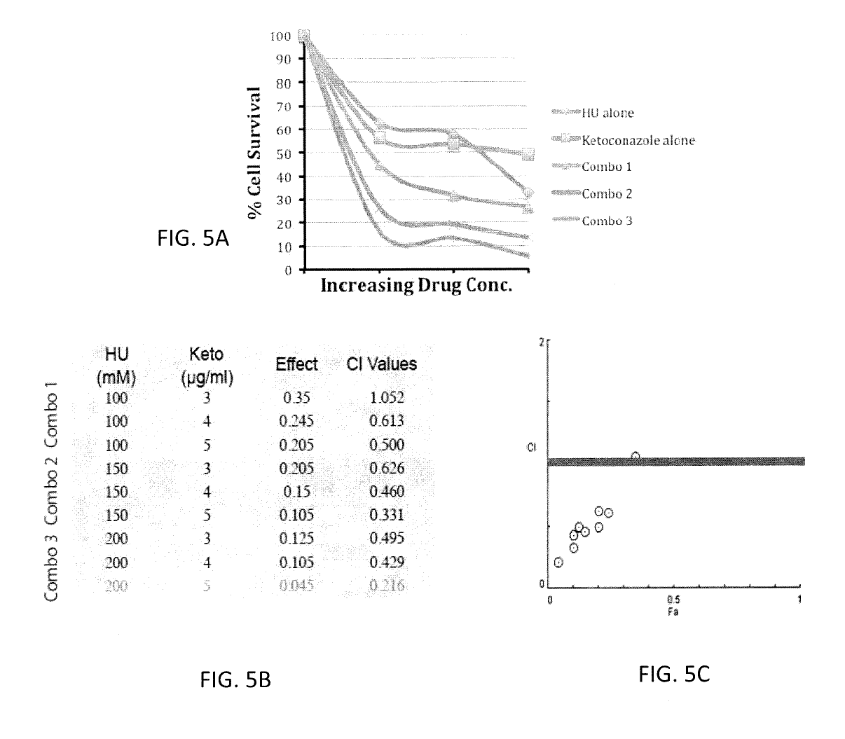

Drug Combinations Used for Testing on Candia albicans in FIG. 5A-C:
Concentration of hydroxyurea used (mM): 0, 100.0, 150.0, 200.0.
Concentration of ketoconazole used (μg/ml): 0, 3.0, 4.0, 5.0.

### Comb 1: (0+0), (100.0+3.0), (100.0+4.0), (100.0+5.0).

### Comb 2: (0+0), (150.0+3.0), (150.0+4.0), (150.0+5.0).

### Comb 3: (0+0), (200.0+3.0), (200.0+4.0), (200.0+5.0).

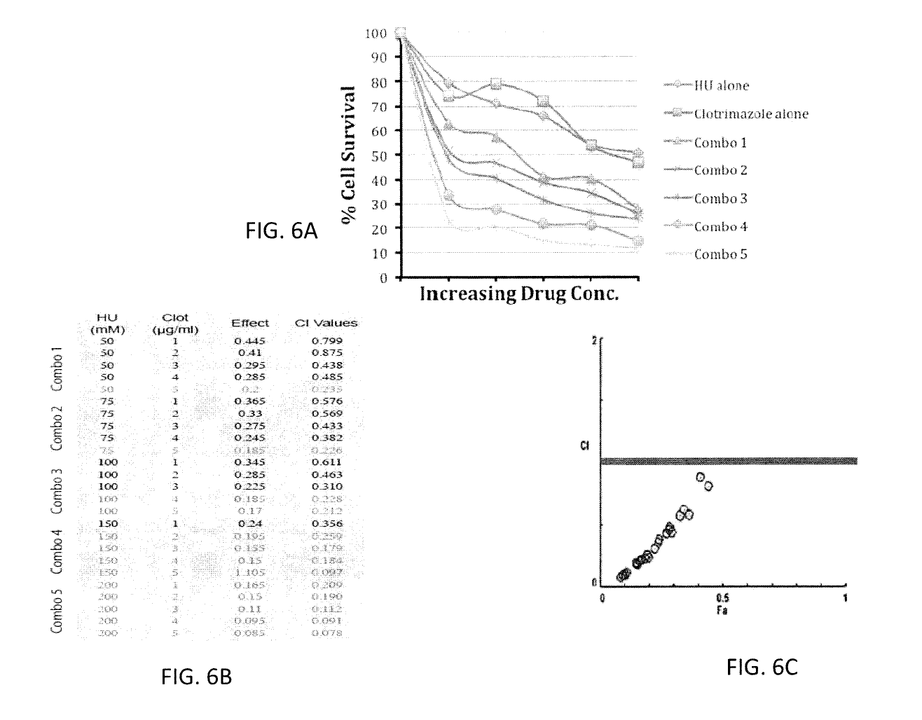

Drug Combinations Used for Testing on Candida. Albicans in FIG. 6A-C:
Concentration of hydroxyurea used (mM): 0, 50.0, 75.0, 100.0, 150.0, 200.0
Concentration of clotrimazole used (μg/ml): 0, 1.0, 2.0, 3.0, 4.0, 5.0

### Comb 1: (0+0), (50.0+1.0), (50.0+2.0), (50.0+3.0), (50.0+4.0), (50.0+5.0).

### Comb 2: (0+0), (75.0+1.0), (75.0+2.0), (75.0+3.0), (75.0+4.0), (75.0+5.0).

### Comb 3: (0+0), (100.0+1.0), (100.0+2.0), (100.0+3.0), (100.0+4.0), (100.0+5.0).

### Comb 4: (0+0), (150.0+1.0), (150.0+2.0), (150.0+3.0), (150.0+4.0), (150.0+5.0).

### Comb 5: (0+0), (200.0+1.0), (200.0+2.0), (200.0+3.0), (200.0+4.0), (200.0+5.0).

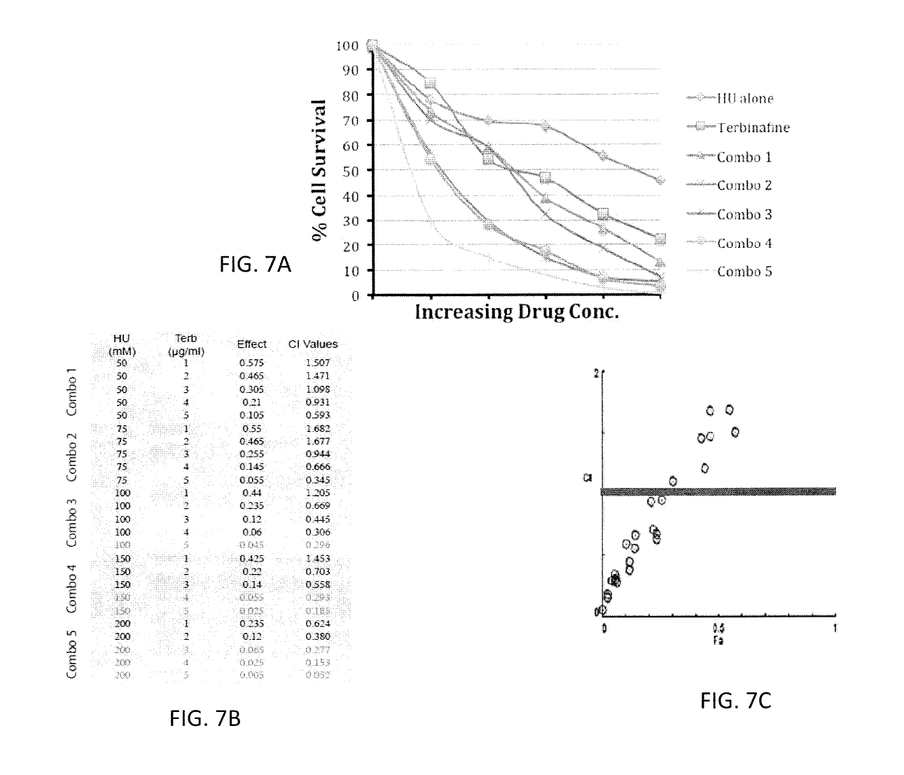

Drug Combinations Used for Testing on Candida albicans in FIG. 7A-C:
Concentration of hydroxyurea used (mM): 0, 50.0, 75.0, 100.0, 150.0, 200.0.
Concentration of terbinafine used (μg/ml): 0, 1.0, 2.0, 3.0, 4.0, 5.0.

### Comb 1: (0+0), (50.0+1.0), (50.0+2.0), (50.0+3.0), (50.0+4.0), (50.0+5.0).

### Comb 2: (0+0), (75.0+1.0), (75.0+2.0), (75.0+3.0), (75.0+4.0), (75.0+5.0).

### Comb 3: (0+0), (100.0+1.0), (100.0+2.0), (100.0+3.0), (100.0+4.0), (100.0+5.0).

### Comb 4: (0+0), (150.0+1.0), (150.0+2.0), (150.0+3.0), (150.0+4.0), (150.0+5.0).

### Comb 5: (0+0), (200.0+1.0), (200.0+2.0), (200.0+3.0), (200.0+4.0), (200.0+5.0).

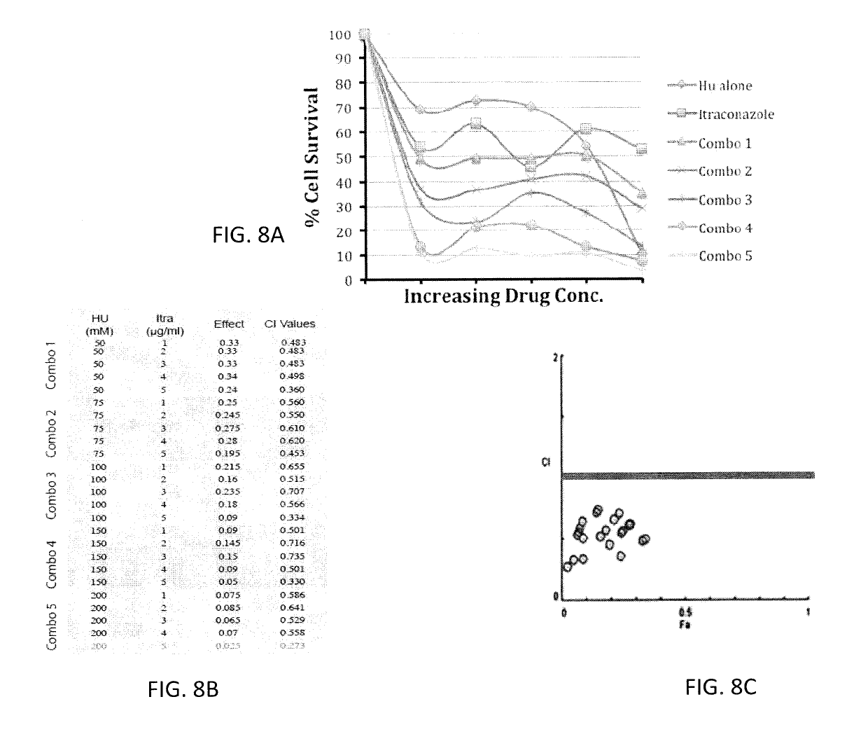

Drug Combinations Used for Testing on Candida albicans in FIG. 8A-C:
Concentration of hydroxyurea used (mM): 0, 50.0, 75.0, 100.0, 150.0, 200.0.
Concentration of itraconazole used (μg/ml): 0, 1.0, 2.0, 3.0, 4.0, 5.0.

### Comb 1: (0+0), (50.0+1.0), (50.0+2.0), (50.0+3.0), (50.0+4.0), (50.0+5.0).

### Comb 2: (0+0), (75.0+1.0), (75.0+2.0), (75.0+3.0), (75.0+4.0), (75.0+5.0).

### Comb 3: (0+0), (100.0+1.0), (100.0+2.0), (100.0+3.0), (100.0+4.0), (100.0+5.0).

### Comb 4: (0+0), (150.0+1.0), (150.0+2.0), (150.0+3.0), (150.0+4.0), (150.0+5.0).

### Comb 5: (0+0), (200.0+1.0), (200.0+2.0), (200.0+3.0), (200.0+4.0), (200.0+5.0).

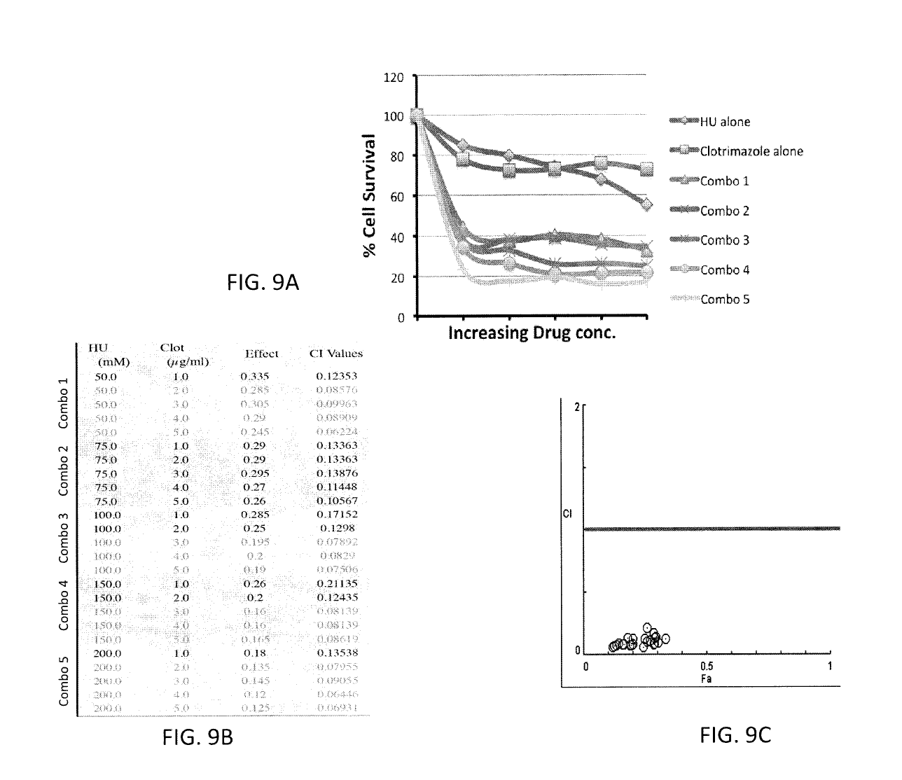

Drug Combinations Used for Testing on Saccharomyces cerevisiae in FIG. 9A-C:
Concentration of hydroxyurea used (mM)—0, 50.0, 75.0, 100.0, 150.0, 200.0.
Concentration of clotrimazole used (μg/ml)—0, 1.0, 2.0, 2.0, 4.0, 5.0.

### Combo 1—(0+0), (50.0+1.0), (50.0+2.0), (50.0+3.0), (50.0+4.0), (50.0+5.0).

### Combo 2—(0+0), (75.0+1.0), (75.0+2.0), (75.0+3.0), (75.0+4.0), (75.0+5.0).

### Combo 3—(0+0), (100.0+1.0), (100.0+2.0), (100.0+3.0), (100.0+4.0), (100.0+5.0).

### Combo 4—(0+0), (150.0+1.0), (150.0+2.0), (150.0+3.0), (150.0+4.0), (150.0+5.0).

### Combo 5—(0+0), (200.0+1.0), (200.0+2.0), (200.0+3.0), (200.0+4.0), (200.0+5.0).

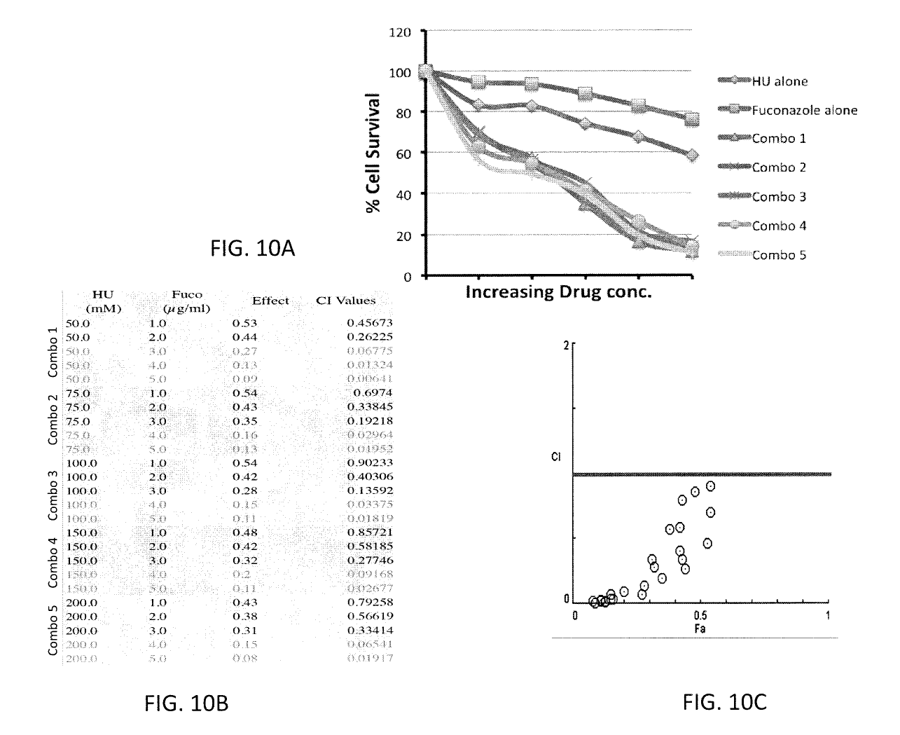

Drug Combinations Used for Testing on Saccharomyces cerevisiae in FIG. 10A-C:
Concentration of hydroxyurea used (mM)—0, 50.0, 75.0, 100.0, 150.0, 200.0.
Concentration of fluconazole used (μg/ml)—0. 1.0, 2.0, 3.0, 4.0, 5.0.

### Combo 1—(0+0), (50.0+1.0), (50.0+2.0), (50.0+3.0), (50.0+4.0), (50.0+5.0).

### Combo 2—(0+0), (75.0+1.0), (75.0+2.0), (75.0+3.0), (75.0+4.0), (75.0+5.0).

### Combo 3—(0+0), (100.0+1.0), (100.0+2.0), (100.0+3.0), (100.0+4.0), (100.0+5.0).

### Combo 4—(0+0), (150.0+1.0), (150.0+2.0), (150.0+3.0), (150.0+4.0), (150.0+5.0).

### Combo 5—(0+0), (200.0+1.0), (200.0+2.0), (200.0+3.0), (200.0+4.0), (200.0+5.0).

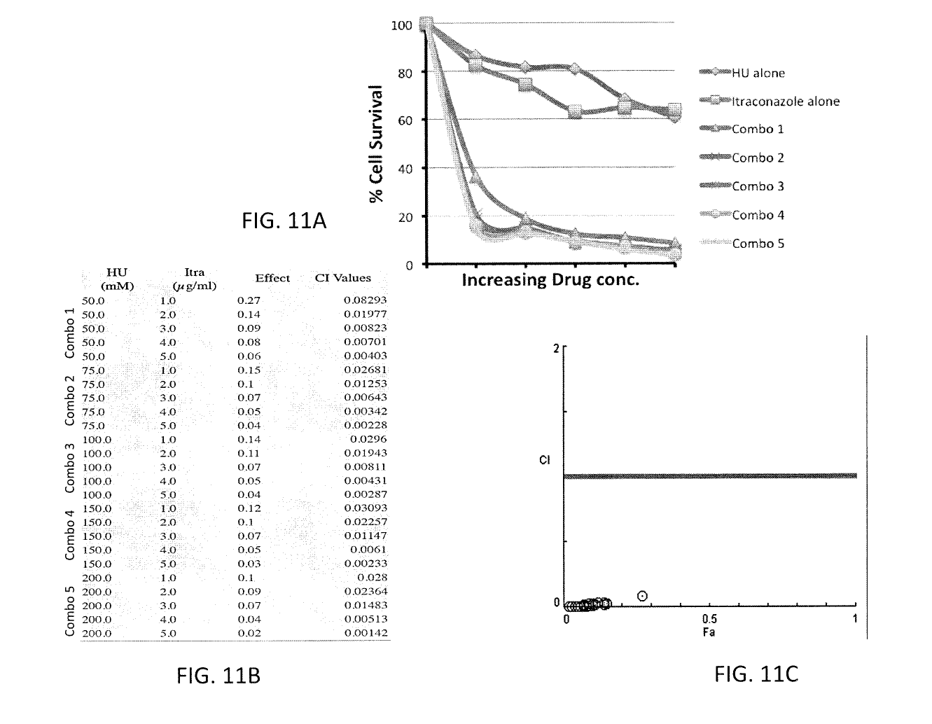

Drug Combinations Used for Testing on Saccharomyces cerevisiae in FIG. 11A-C:
Concentration of hydroxyurea used (mM)—0, 50.0, 75.0, 100.0, 150.0, 200.0.
Concentration of itraconazole used (μg/ml)—0. 1.0, 2.0, 3.0, 4.0, 5.0.

### Combo 1—(0+0), (50.0+1.0), (50.0+2.0), (50.0+3.0), (50.0+4.0), (50.0+5.0).

### Combo 2—(0+0), (75.0+1.0), (75.0+2.0), (75.0+3.0), (75.0+4.0), (75.0+5.0).

### Combo 3—(0+0), (100.0+1.0), (100.0+2.0), (100.0+3.0), (100.0+4.0), (100.0+5.0).

### Combo 4—(0+0), (150.0+1.0), (150.0+2.0), (150.0+3.0), (150.0+4.0), (150.0+5.0).

### Combo 5—(0+0), (200.0+1.0), (200.0+2.0), (200.0+3.0), (200.0+4.0), (200.0+5.0).

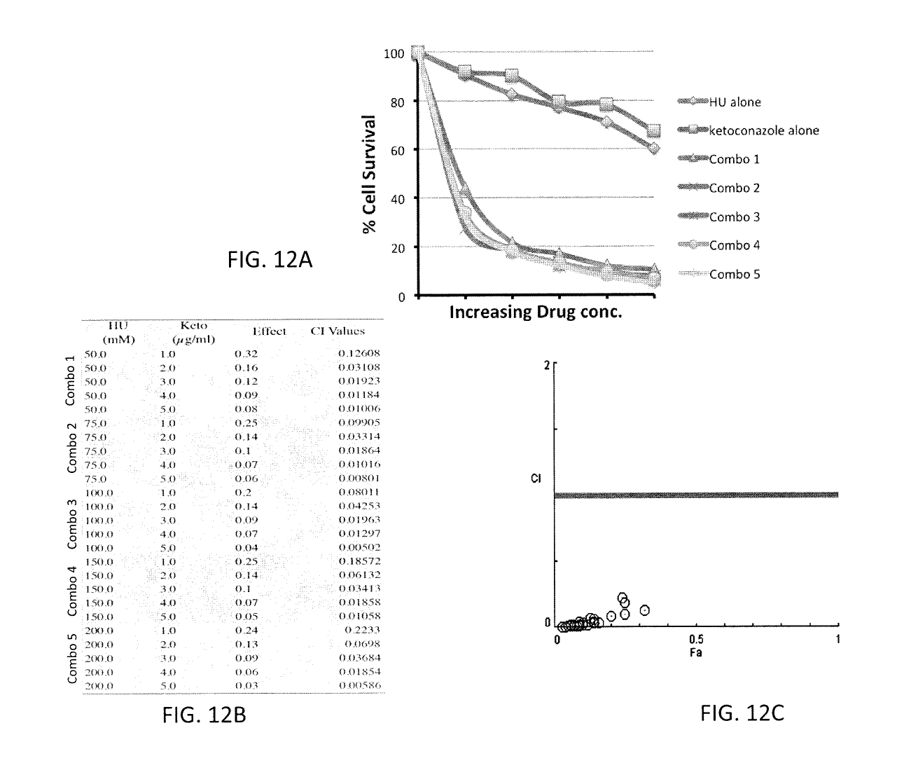

Drug Combinations Used for Testing on Saccharomyces cerevisiae in FIG. 12A-C:
Concentration of hydroxyurea used (mM)—0, 50.0, 75.0, 100.0, 150.0, 200.0.
Concentration of ketoconazole used (μg/ml)—0. 1.0, 2.0, 3.0, 4.0, 5.0.

### Combo 1—(0+0), (50.0+1.0), (50.0+2.0), (50.0+3.0), (50.0+4.0), (50.0+5.0).

### Combo 2—(0+0), (75.0+1.0), (75.0+2.0), (75.0+3.0), (75.0+4.0), (75.0+5.0).

### Combo 3—(0+0), (100.0+1.0), (100.0+2.0), (100.0+3.0), (100.0+4.0), (100.0+5.0).

### Combo 4—(0+0), (150.0+1.0), (150.0+2.0), (150.0+3.0), (150.0+4.0), (150.0+5.0).

### Combo 5—(0+0), (200.0+1.0), (200.0+2.0), (200.0+3.0), (200.0+4.0), (200.0+5.0).

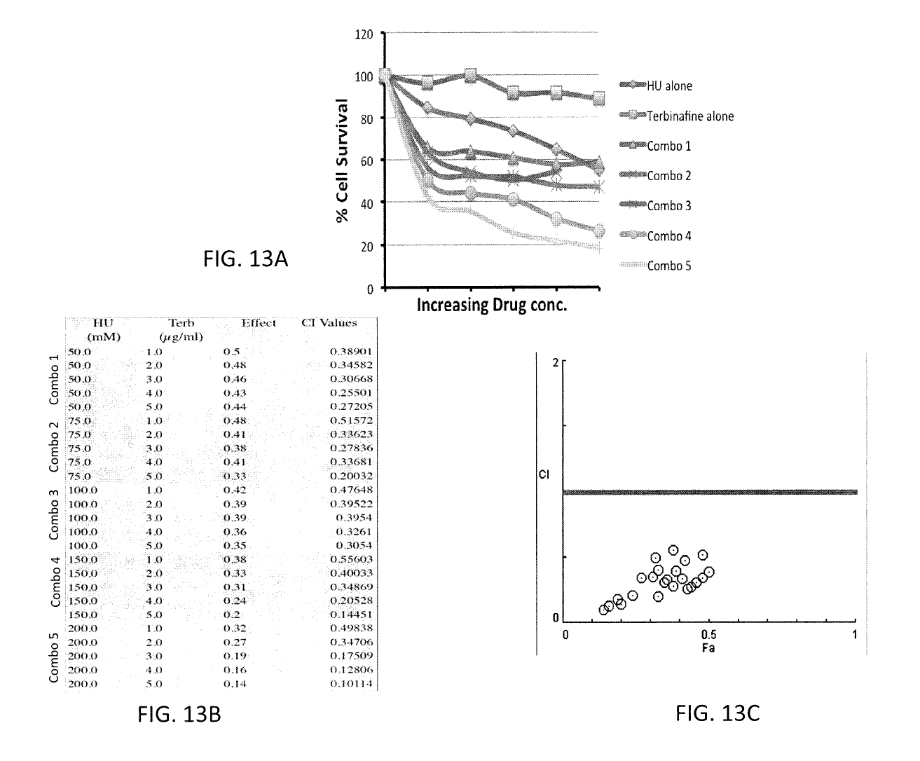

Drug Combinations Used for Testing on Saccharomyces cerevisiae in FIG. 13A-C:
Concentration of hydroxyurea used (mM)—0, 50.0, 75.0, 100.0, 150.0, 200.0.
Concentration of terbinafine used (μg/ml)—0. 1.0, 2.0, 3.0, 4.0, 5.0.

### Combo 1—(0+0), (50.0+1.0), (50.0+2.0), (50.0+3.0), (50.0+4.0), (50.0+5.0).

### Combo 2—(0+0), (75.0+1.0), (75.0+2.0), (75.0+3.0), (75.0+4.0), (75.0+5.0).

### Combo 3—(0+0), (100.0+1.0), (100.0+2.0), (100.0+3.0), (100.0+4.0), (100.0+5.0).

### Combo 4—(0+0), (150.0+1.0), (150.0+2.0), (150.0+3.0), (150.0+4.0), (150.0+5.0).

### Combo 5—(0+0), (200.0+1.0), (200.0+2.0), (200.0+3.0), (200.0+4.0), (200.0+5.0).

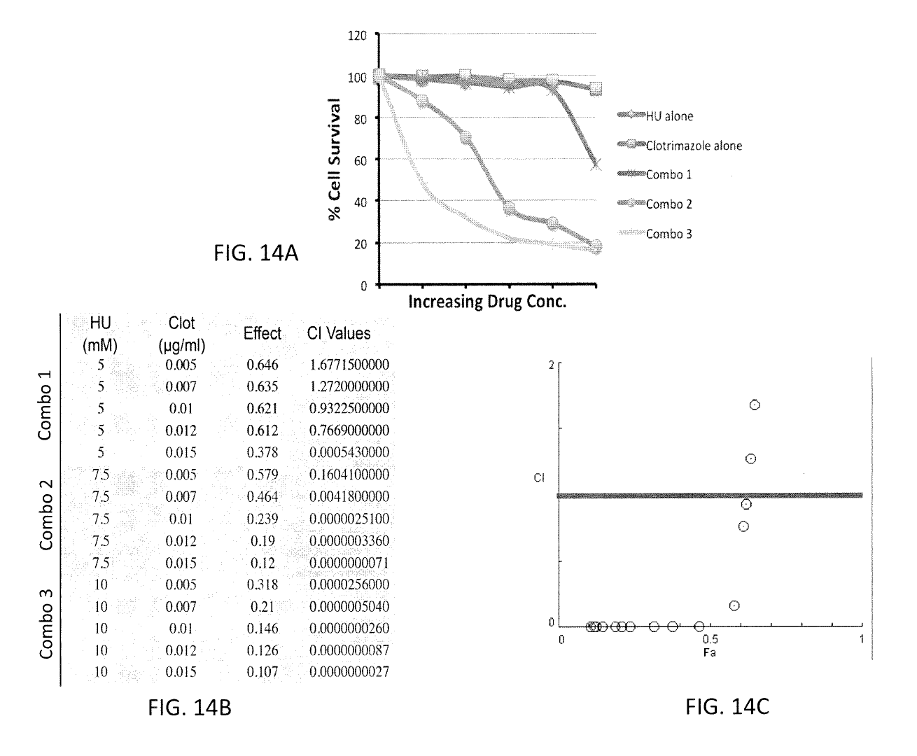

Drug Combinations Used for Testing on Schizosaccharomyces pombe in FIG. 14A-C:
Concentration of hydroxyurea used (mM)—0, 5.0, 7.5, 10.0.
Concentration of clotrimazole used (μg/ml)—0, 0.005, 0.007, 0.01, 0.012, 0.015.

### Combo 1—(0+0), (5.0+0.005), (5.0+0.007), (5.0+0.01), (5.0+0.012), (5.0+0.015).

### Combo 2—(0+0), (7.5+0.005), (7.5+0.007), (7.5+0.01), (7.5+0.012), (7.5+0.015).

### Combo 3—(0+0), (10.0+0.005), (10.0+0.007), (10.0+0.01), (10.0+0.012), (10.0+0.015).

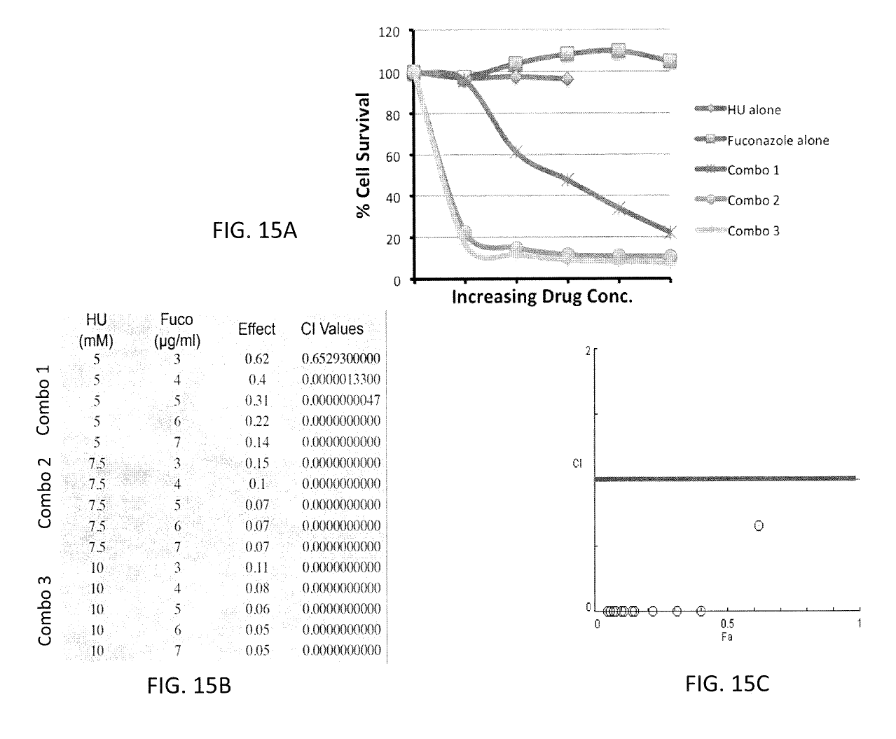

Drug Combinations Used for Testing on Schizosaccharomyces pombe in FIG. 15A-C:
Concentration of hydroxyurea used (mM)—0, 5.0, 7.5, 10.0.
Concentration of fluconazole used (μg/ml)—0, 3.0, 4.0, 5.0, 6.0, 7.0.

### Combo 1—(0+0), (5.0+3.0), (5.0+4.0), (5.0+5.0), (5.0+6.0), (5.0+7.0).

### Combo 2—(0+0), (7.5+3.0), (7.5+4.0), (7.5+5.0), (7.5+6.0), (7.5+7.0).

### Combo 3—(0+0), (10.0+3.0), (10.0+4.0), (10.0+5.0), (10.0+6.0), (10.0+7.0).

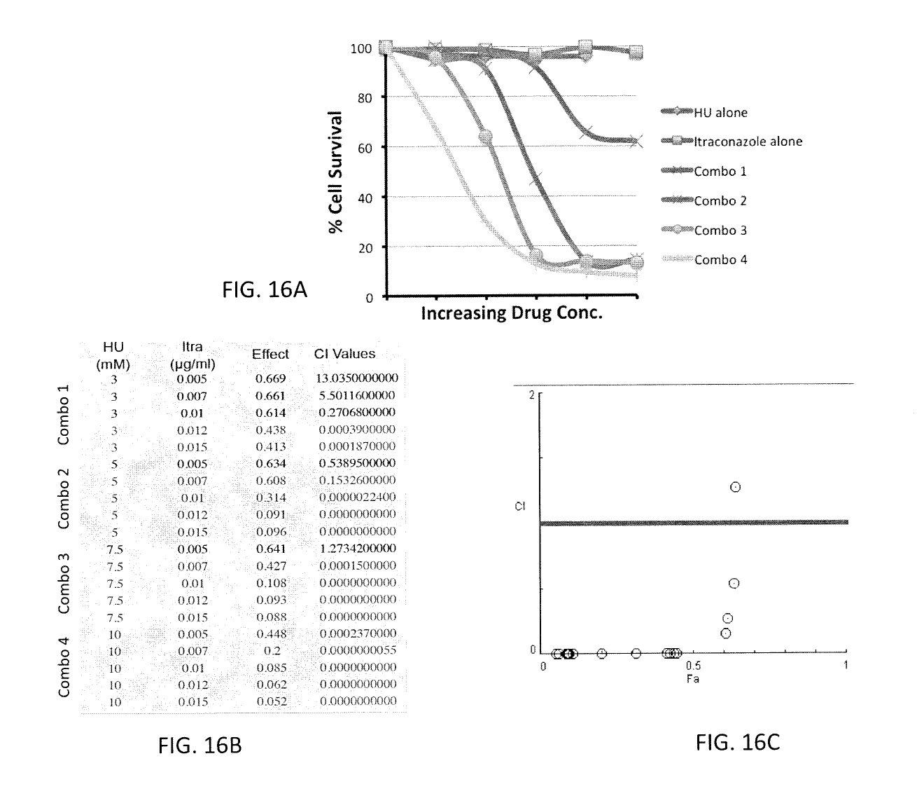

Drug Combinations Used for Testing on Schizosaccharomyces pombe in FIG. 16A-C:
Concentration of hydroxyurea used (mM)—0, 3.0, 5.0, 7.5, 10.0.
Concentration of itraconazole used (μg/ml)—0, 0.005, 0.007, 0.01, 0.012, 0.015.

### Combo 1—(0+0), (3.0+0.005), (3.0+0.007), (3.0+0.01), (3.0+0.012), (3.0+0.015).

### Combo 2—(0+0), (5.0+0.005), (5.0+0.007), (5.0+0.01), (5.0+0.012), (5.0+0.015).

### Combo 3—(0+0), (7.5+0.005), (7.5+0.007), (7.5+0.01), (7.5+0.012), (7.5+0.015).

### Combo 4—(0+0), (10.0+0.005), (10.0+0.007), (10.0+0.01), (10.0+0.012), (10.0+0.015).

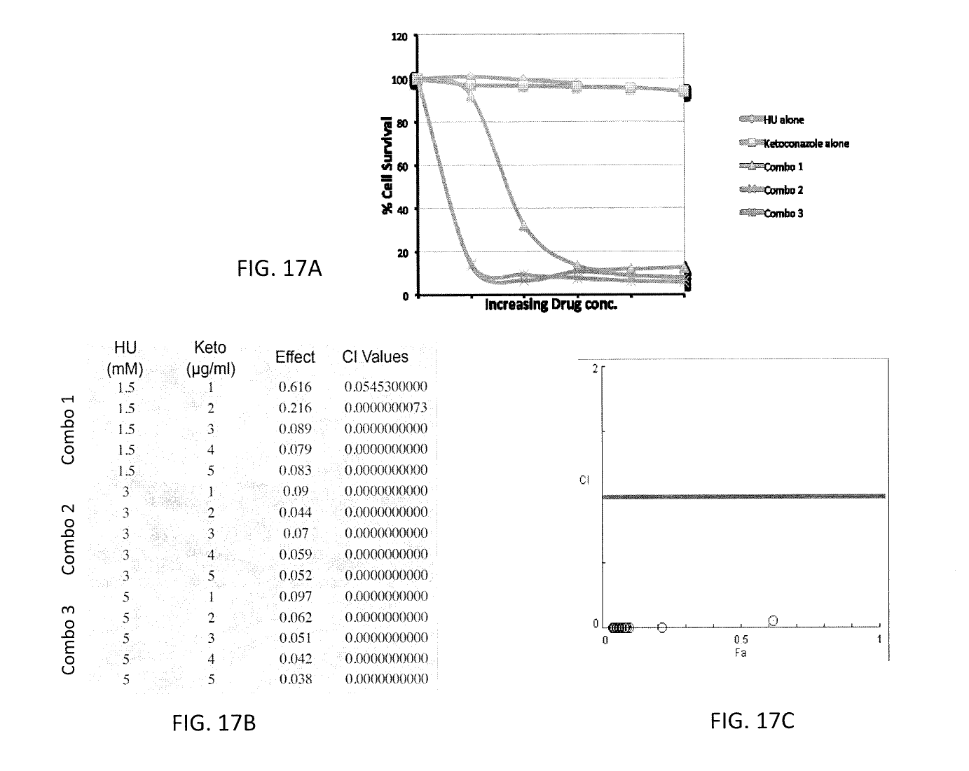

Drug Combinations Used for Testing on Schizosaccharomyces pombe in FIG. 17A-C:
Concentration of hydroxyurea used (mM)—0, 1.5, 3.0, 5.0.
Concentration of ketoconazole used (μg/ml)—0, 1.0, 2.0, 3.0, 4.0, 5.0.

### Combo 1—(0+0), (1.5+1.0), (1.5+2.0), (1.5+3.0), (1.5+4.0), (1.5+5.0).

### Combo 2—(0+0), (3.0+1.0), (3.0+2.0), (3.0+3.0), (3.0+4.0), (3.0+5.0).

### Combo 3—(0+0), (5.0+1.0), (5.0+2.0), (5.0+3.0), (5.0+4.0), (5.0+5.0).

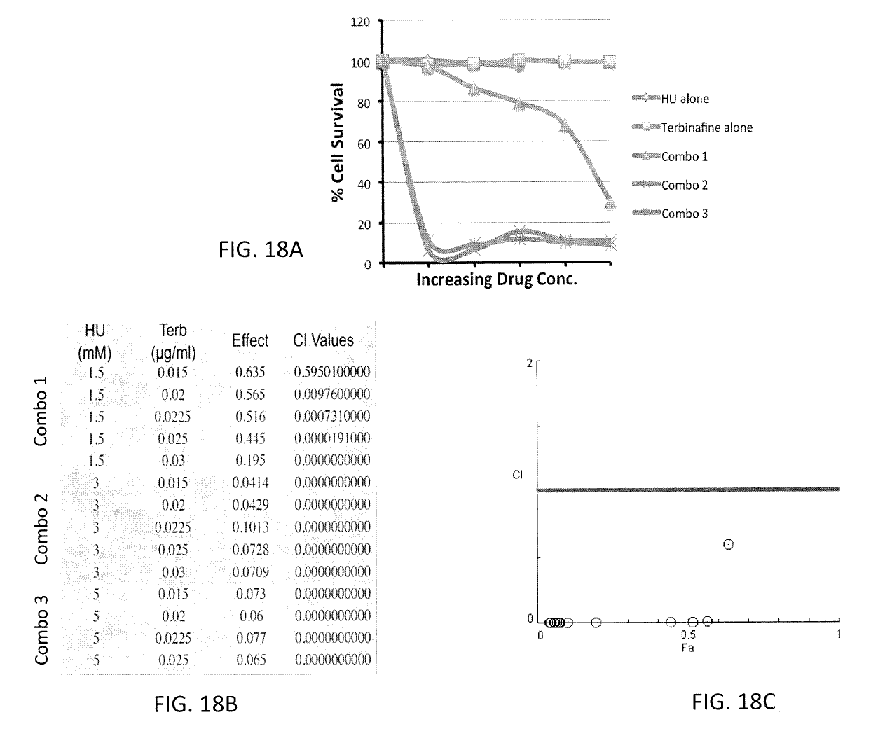

Drug Combinations Used for Testing on Schizosaccharomyces pombe in FIG. 18A-C:
Concentration of hydroxyurea used (mM)—1, 1.5, 3.0, 5.0.
Concentration of terbinafine used (μg/ml)—0, 0.015, 0.02, 0.0225, 0.025, 0.03.

### Comb 1—(0+0), (1.5+0.015), (1.5+0.02), (1.5+0.0225), (1.5+0.025), (1.5+0.03).

### Comb 2—(0+0), (1.5+0.015), (1.5+0.02), (1.5+0.0225), (1.5+0.025), (1.5+0.03).

### Comb 3—(0+0), (1.5+0.015), (1.5+0.02), (1.5+0.0225), (1.5+0.025), (1.5+0.03).

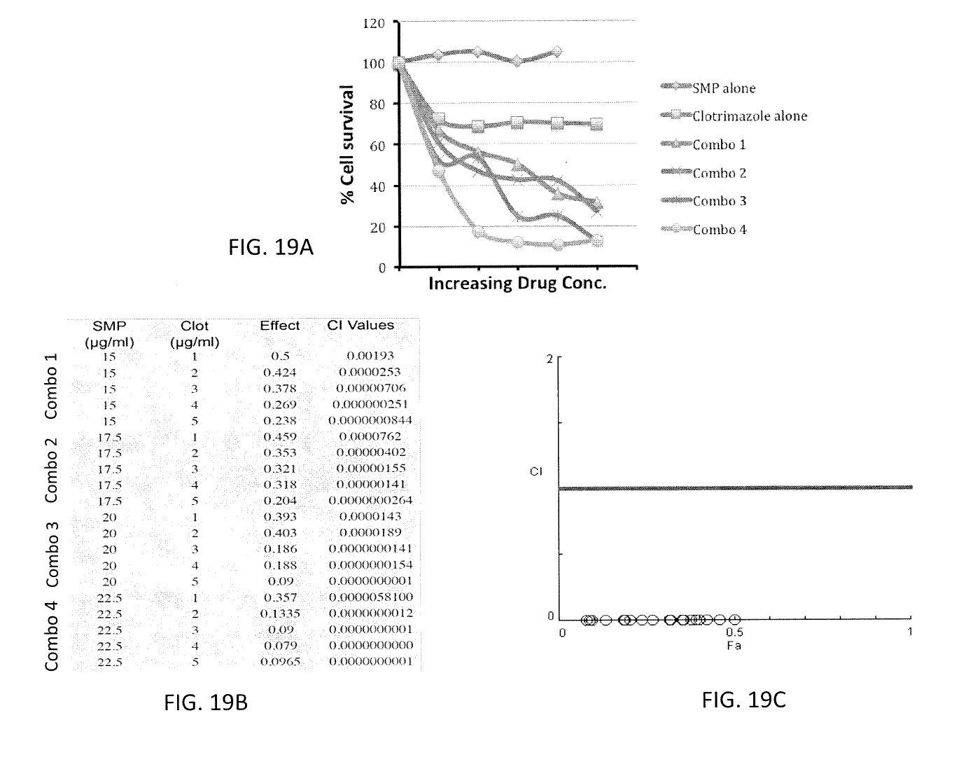

Drug Combinations Used for Testing on Candida albicans in FIG. 19A-C:
Concentration of sampangine used (μg/ml): 0, 15.0, 17.5, 20.0, 22.5.
Concentration of clotrimazole used (μg/ml): 0, 1.0, 2.0, 3.0, 4.0, 5.0.

### Combo 1—(0+0), (15.0+1.0), (15.0+2.0), (15.0+3.0), (15.0+4.0), (15.0+5.0).

### Combo 2—(0+0), (17.5+1.0), (17.5+2.0), (17.5+3.0), (17.5+4.0), (17.5+5.0).

### Combo 3—(0+0), (20.0+1.0), (20.0+2.0), (20.0+3.0), (20.0+4.0), (20.0+5.0).

### Combo 4—(0+0), (22.5+1.0), (22.5+2.0), (22.5+3.0), (22.5+4.0), (22.5+5.0).

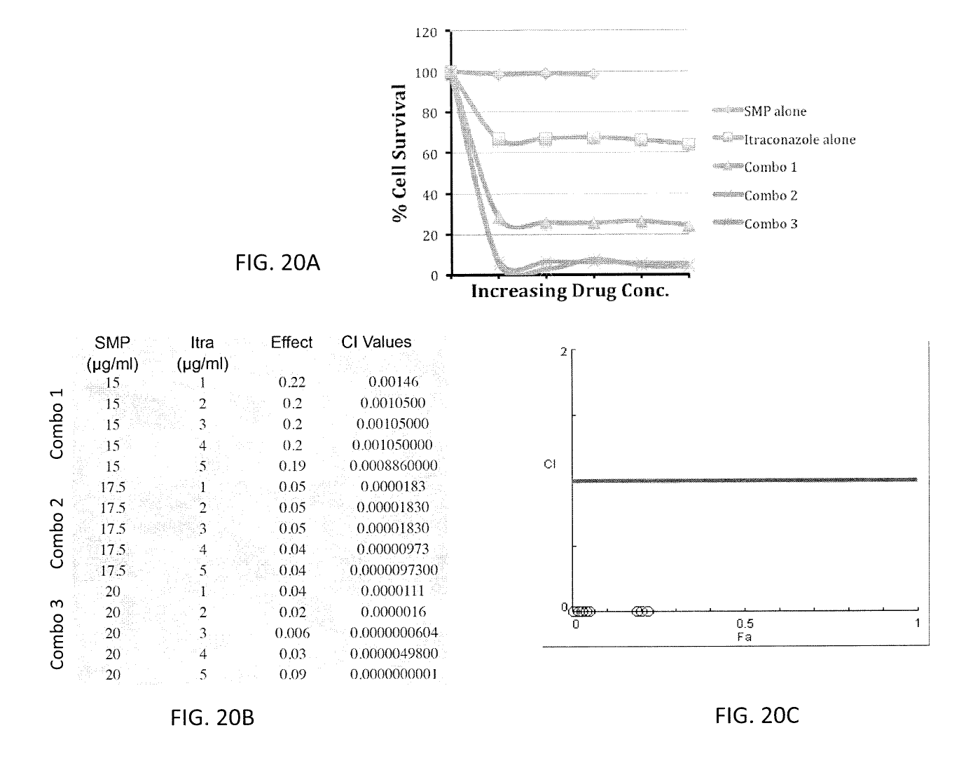

Drug Combinations Used for Testing on Candida albicans in FIG. 20A-C:
Concentration of sampangine used (μg/ml): 0, 15.0, 17.5, 20.0.
Concentration of itraconazole used (μg/ml): 0, 1.0, 2.0, 3.0, 4.0, 5.0.

### Combo 1—(0+0), (15.0+1.0), (15.0+2.0), (15.0+3.0), (15.0+4.0), (15.0+5.0).

### Combo 2—(0+0), (17.5+1.0), (17.5+2.0), (17.5+3.0), (17.5+4.0), (15.75+5.0).

### Combo 3—(0+0), (20.0+1.0), (20.0+2.0), (20.0+3.0), (20.0+4.0), (20.0+5.0).

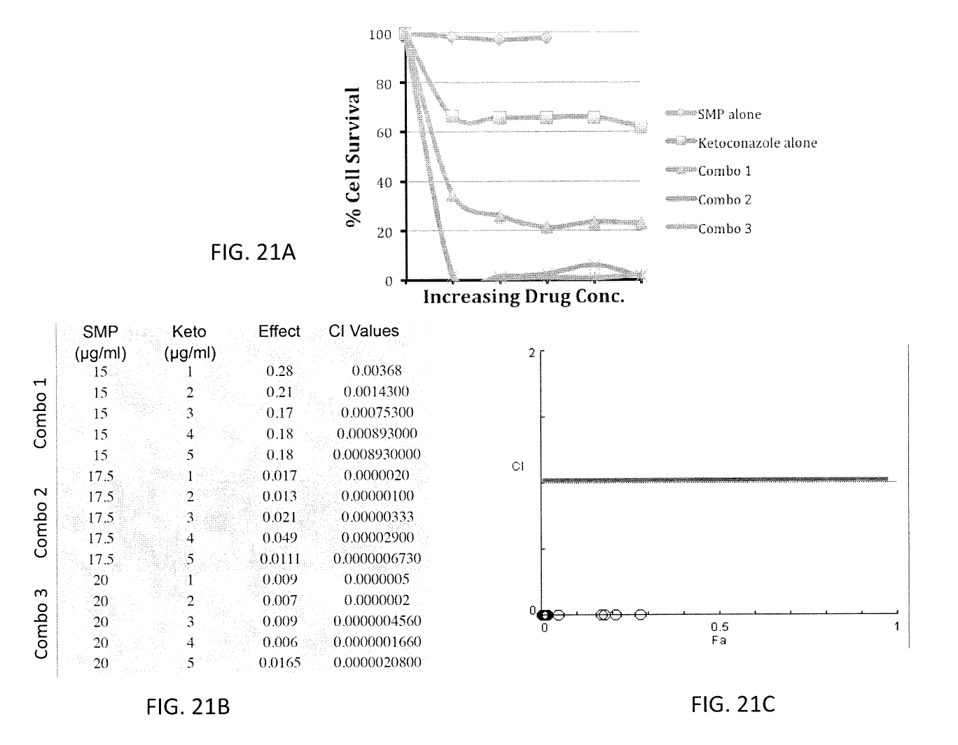

Drug Combinations Used for Testing on Candida albicans in FIG. 21A-C:
Concentration of sampangine used (μg/ml): 0, 15.0, 17.5, 20.0.
Concentration of ketoconazole used (μg/ml): 0, 1.0, 2.0, 3.0, 4.0, 5.0.

### Combo 1—(0+0), (15.0+1.0), (15.0+2.0), (15.0+3.0), (15.0+4.0), (15.0+5.0).

### Combo 2—(0+0), (17.5+1.0), (17.5+2.0), (17.5+3.0), (17.5+4.0), (15.75+5.0).

### Combo 3—(0+0), (20.0+1.0), (20.0+2.0), (20.0+3.0), (20.0+4.0), (20.0+5.0).

### EXEMPLARY RESULTS AND DISCUSSION

To analyze the synergistic drug effect in detail, the present investigators measured the cytotoxicity in 96 well plates as described in Materials and Methods. The dose response curve of each drug was obtained in order to determine the drug concentration required to suppress 50% cell growth (IC50 value). The drug concentrations showing minimal effects on cell growth were then chosen for various drug combinations. The synergistic, additive, or antagonistic effects between the combined drugs are determined by using the Chou and Talalay method. As shown in FIG. 2A-C, surprisingly most of the combinations of sampangine and hydroxyurea showed a synergistic cell killing effect on wildtype Schizosaccharomyces pombe of as the calculated combination index (CI) values are less than 1.0 (FIG. 2C). Although a few combinations showed an additive (CI values 1.0) or antagonistic (CI values >1.0) effect, more than half of the combinations showed a very strong synergism (CI values <0.1) (FIG. 2B). These data are consistent with the results from FIG. 1A-C and suggests that similar synergistic effects may be observed with other fungi, particularly the pathogenic fungi such as Candida albicans.

To test this hypothesis, the present investigators first assessed the effect of sampangine and hydroxyurea on the budding yeast Saccharomyces cerevisiae, another non-pathogenic fungus (FIG. 3A-C). Similar synergistic effects were observed in a wide range of drug combinations although higher concentrations of hydroxyurea are used because it is known that Saccharomyces cerevisiae is more resistant to the agent. The present investigators then tested the effect of hydroxyurea and sampangine on Candida albicans (FIG. 4A-C). Again, surprisingly most of the combinations showed a strongly synergistic effect. Similar to Saccharomyces cerevisiae, higher concentrations of hydroxyurea are required to achieve the dramatic cell-killing effect on Candida albicans.

The present investigators then tested the effect of hydroxyurea in combination with four ergosterol synthesis inhibitors on Candida albicans (FIG. 5A-C through FIG. 8A-C). When hydroxyurea was combined with ketoconazole (FIG. 5A-C), the combination showed a synergistic effect only when higher concentrations of hydroxyurea of between 100 and 200 mM were used. When clotrimazole was combined with hydroxyurea (FIG. 6A-C), some of the combinations were strongly synergistic and the concentration of hydroxyurea can be lowered to less than 50 mM. While terbinafine showed a similar effect as ketoconazole, in which a higher concentration of hydroxyurea is required (FIG. 7A-C), itraconazole showed an effect similar to that observed with clotrimazole (FIG. 8A-C).

The present investigators further tested the effect of hydroxyurea in combination with five ergosterol synthesis inhibitors on Saccharomyces cerevisiae (FIG. 9A-C through FIG. 13A-C). When hydroxyurea was combined with clotrimazole (FIG. 9A-C), most combinations demonstrated a very strong synergistic effect, while combinations with 75 mM of hydroxyurea showed a strong synergistic effect. When hydroxyurea was combined with fluconazole (FIG. 10A-C), the combination showed a very strong synergistic effect at higher levels of fluconazole for every level of hydroxyurea tested. When hydroxyurea was combined with itraconazole (FIG. 11A-C), all combinations demonstrated a very strong synergistic effect. Similarly, when hydroxyurea was combined with ketoconazole (FIG. 12A-C), almost all combinations demonstrated a very strong synergistic effect. Terbinafine (FIG. 13A-C) showed a similar effect to fluconazole, in that the combination showed a strong synergistic effect at higher levels of terbinafine for every level of hydroxyurea tested.

The present investigators further tested the effect of hydroxyurea in combination with five ergosterol synthesis inhibitors on Schizosaccharomyces pombe (FIG. 14A-C through FIG. 18A-C). When hydroxyurea was combined with clotrimazole (FIG. 14A-C), the combination showed a synergistic effect only when higher concentrations of hydroxyurea of between 7.5 and 10 mM were used. When hydroxyurea was combined with fluconazole (FIG. 15A-C), the combination showed a very strong synergistic effect at almost all combinations tested. Similarly, when hydroxyurea was combined with itraconazole (FIG. 16A-C), almost all combinations demonstrated a very strong synergistic effect. When hydroxyurea was combined with ketoconazole (FIG. 17A-C), all combinations demonstrated a very strong synergistic effect. Terbinafine (FIG. 18A-C) showed a similar effect to fluconazole and itraconazole, in that the combination showed a very strong synergistic effect at almost all combinations tested.

The present investigators further tested the effect of a sampangine in combination with ergosterol synthesis inhibitors on Candida albicans (FIG. 19A-C through FIG. 21A-C). When sampangine was combined with clotrimazole (FIG. 19A-C), itraconazole (FIG. 20A-C), or ketoconazole (FIG. 21A-C), surprisingly all combinations demonstrated a very strong synergistic effect.

Hydroxyurea is a non-alkylating antiproliferative and antiviral agent that has been used for over several decades to treat a variety of neoplastic and non-neoplastic conditions. It is water-soluble, readily absorbed, and widely distributed throughout the body. Hydroxyurea is less toxic as compared with other chemotherapeutic agents. It is tolerable to patients when applied onto the skin at the concentrations of 10% (1.3 M) to 15% (2.0 M). Based on the instant findings, the immediate implication is that hydroxyurea can significantly improve the therapeutic effect of various azoles for fungal infections on skin, nails, or mucosa. Since certain species of fungi such as Schizosaccharomyces pombe are highly sensitive to hydroxyurea, we expect that some pathogenic fungi may also be sensitive to this agent, which may expand the use of hydroxyurea in invasive or chronic fungal infections. This may include other known species of pathogenic fungi such as Aspergillus fumigatus, Histoplasma capsulatum, and Cryptococcus neoformans, which would allow for more efficient treatments with less toxicity. Apart from treating fungal infections, our combination therapy using hydroxyurea may also have the potential to treat certain types of cancers, because hydroxyurea has already been used in cancer chemotherapy. One of the azoles, itraconazole, has been used for treating patients with prostate cancer. Together, our discovery using hydroxyurea in combination with sampangine and the ergosterol synthesis inhibitors, as well as the combination of sampangine and the ergosterol synthesis inhibitors, may have a great therapeutic potential for the treatment of fungal infections and cancers.

1-19. (canceled)

20. A method of treating a fungal infection in a subject in need thereof, the method comprising administering to the subject a therapeutically effective amount of hydroxyurea and a therapeutically effective amount of a heme synthesis inhibitor.

21. The method of claim 20 wherein the fungal infection involves a fungal species comprising Candida, Aspergillus, Cryptococcus, Pneumocystis, Histoplasma, and Cryptococcus species.

22. The method of claim 20, wherein the heme synthesis inhibitor comprises sampangine.

23. The method of claim 21, wherein said fungal species is Candida albicans.

24. The method of claim 21, wherein said fungal species is Aspergillus fumigatus.

25. The method of claim 21, wherein said fungal species is Histoplasma capsulatum.

26. The method of claim 21, wherein said fungal species is Cryptococcus neoformans.

27. A pharmaceutical composition comprising:
a therapeutically effective amount of hydroxyurea;
a therapeutically effective amount of a heme synthesis inhibitor;
and a pharmaceutically-acceptable excipient.

28. The pharmaceutical composition of claim 27, wherein the heme synthesis inhibitor comprises sampangine.

29. A method of treating a fungal infection in a subject in need thereof, the method comprising administering to the subject a therapeutically effective amount of an ergosterol synthesis inhibitor and a therapeutically effective amount of a heme synthesis inhibitor.

30. The method of claim 29 wherein the fungal infection involves a fungal species comprising Candida, Aspergillus, Cryptococcus, Pneumocystis, Histoplasma, and Cryptococcus species.

31. The method of claim 29, wherein the ergosterol synthesis inhibitor comprises an azole.

32. The method of claim 31, wherein the ergosterol synthesis inhibitor comprises ketoconazole.

33. The method of claim 31, wherein the ergosterol synthesis inhibitor comprises clotrimazole.

34. The method of claim 31, wherein the ergosterol synthesis inhibitor comprises intraconazole.

35. The method of claim 31, wherein the ergosterol synthesis inhibitor comprises fluconazole.

36. The method of claim 29, wherein the ergosterol synthesis inhibitor comprises terbinafine.

37. The method of claim 29, wherein the heme synthesis inhibitor comprises sampangine.

38. The method of claim 30, wherein said fungal species is Candida albicans.

39. The method of claim 30, wherein said fungal species is Aspergillus fumigatus.

40. The method of claim 30, wherein said fungal species is Histoplasma capsulatum.

41. The method of claim 30, wherein said fungal species is Cryptococcus neoformans.

42. A pharmaceutical composition comprising:
a therapeutically effective amount of an ergosterol synthesis inhibitor;
a therapeutically effective amount of a heme synthesis inhibitor;
and a pharmaceutically-acceptable excipient.

43. The pharmaceutical composition of claim 42, wherein the ergosterol synthesis inhibitor comprises an azole.

44. The pharmaceutical composition of claim 43, wherein the ergosterol synthesis inhibitor comprises ketoconazole.

45. The pharmaceutical composition of claim 43, wherein the ergosterol synthesis inhibitor comprises clotrimazole.

46. The pharmaceutical composition of claim 43, wherein the ergosterol synthesis inhibitor comprises intraconazole.

47. The pharmaceutical composition of claim 42, wherein the ergosterol synthesis inhibitor comprises terbinafine.

48. The pharmaceutical composition of claim 43, wherein the ergosterol synthesis inhibitor comprises fluconazole.

49. The pharmaceutical composition of claim 42, wherein the heme synthesis inhibitor comprises sampangine.
